SEQUENCE LISTING (1 sequence)

SEQ ID NO: 1 - protein, 87 aa, Homo sapiens
MSQAEFEKAAEEVRHLKTKPSDEEMLFIYGHYKQATVGDINTERPGMLDFTGKAKWDAWNELKGTSKEDAMKAYINKVEELKKKYGI

(19) United States
(12) Patent Application Publication
(10) Pub. No.: US 2025/0051430 A1
(43) Pub. Date: Feb. 13, 2025

(54) NEUTRALIZATION OF ACYL-COA BINDING PROTEIN CONFERS AUTOPHAGY-DEPENDENT ORGAN PROTECTION

(71) Applicants: Institut National De La Santé Et De La Recherche Médicale, Paris (FR); Assistance Publique-Hôpitaux De Paris, Paris (FR); Sorbonne Université, Paris (FR); Université Paris Cité, Paris (FR)

(72) Inventors: Guido KROEMER, Paris (FR); Omar MOTIÑO GARCIA-MIGUEL, Gunsbourg (FR)

(21) Appl. No.: 18/798,145

(22) Filed: Aug. 8, 2024

Related U.S. Application Data
(63) Continuation of application No. PCT/IB2023/051086, filed on Feb. 7, 2023.

(30) Foreign Application Priority Data
Feb. 8, 2022 (EP) 22305137

(51) Int. Cl. C07K 16/18; A61K 39/00; A61P 1/16

(52) U.S. Cl. CPC C07K 16/18; A61P 1/16; A61K 2039/505; C07K 2317/76

(57) ABSTRACT

Insults to vital organs have serious and even life-threatening consequences. Organ insults have different etiologies and typically include drugs, toxins and ischemic insults. Acyl-CoA binding protein (ACBP), also known as diazepam-binding inhibitor (DBI), is an extracellular feedback regulator of autophagy. Here, the inventors report that injection of a monoclonal antibody neutralizing ACBP/DBI (α-DBI) protects the murine liver against ischemia/reperfusion damage, acute intoxication by acetaminophen and concanavalin A, as well as against liver fibrosis induced by bile duct ligation or carbon tetrachloride. Of note, the results support the contention that α-DBI mediates broad organ-protective effects against multiple insults. Thus, the present invention relates to methods and pharmaceutical composition of protecting organs from injuries comprising neutralization of Acyl-CoA Binding Protein.

### CROSS REFERENCE

This application is a continuation of International Application No. PCT/IB2023/051086, filed Feb. 7, 2023, which claims priority to European Application Serial No. EP22305137, filed Feb. 8, 2022, the disclosures of which are incorporated herein by reference in their entirety.

### SEQUENCE LISTING

The instant application contains a Sequence Listing which has been submitted electronically in XML file format and is hereby incorporated by reference in its entirety. Said XML copy, created on Oct. 25, 2024, is named 206008-702301_SL.xml and is 2,052 bytes in size.

### FIELD OF THE INVENTION

The present invention is in the field of medicine, in particular physiology.

### BACKGROUND OF THE INVENTION

Insults to vital organs, such as, for example, the heart, brain, lungs, kidneys, gastrointestinal tract, or liver, have serious and even life-threatening consequences. Organ insults have different etiologies and typically include drugs, toxins and ischemic insults. For instance, investigations of cellular pathophysiology of ischemic injury in acute myocardial infarction have consistently shown that a significant part of tissue damage occurs during reperfusion, the period when blood flow is restored after an ischemic period of more than about ten minutes. This injury is responsible for paradoxical organ death and tissue injury after termination of the reperfusion period. IRI can be shown in almost all organ systems. The mechanisms involved in IRI include a reduction in high energy phosphate (ATP) levels for several hours after tissue ischemia, inflammatory cell (neutrophils) mediated cellular and microvascular injuries, no reflow phenomena (inadequate reperfusion), microvascular dysfunction with platelet plugging and endothelial damage with inadequate tissue perfusion during the reperfusion period, and calcium overload mediated reperfusion injury.

The common outcome of organ insults is fibrosis. Fibrosis is indeed the formation of fibrous connective tissue in response to injury. It is characterized by the accumulation of extracellular matrix components, particularly collagen, at the site of injury. Fibrosis is an adaptive response that is a vital component of wound healing and tissue repair. However, its continued activation is highly detrimental and a common final pathway of numerous disease states including neuronal, renal, cardiovascular, liver and respiratory diseases.

Thus, a need remains therapies for protecting organs from injuries and thus for subsequently preventing fibrosis.

Macroautophagy (hereafter referred to as ‘autophagy’) is a process through which portions of the cytoplasm are sequestered in autophagosomes, which subsequently fuse with lysosomes for the enzymatic hydrolysis of the autophagic cargo (Morishita and Mizushima, 2019). Although autophagy is often observed in the context of cell death, it preponderantly subserves cytoprotective functions. Thus, excessive autophagy leading to cellular demise (‘autophagic cell death’ or ‘autosis’) is a rare phenomenon. Rather, in most instances, cell stress-induced autophagy delays or avoids cell death by facilitating cellular adaptation (Kroemer and Levine, 2008; López-Otin and Kroemer, 2021; Schwartz, 2021). This stress-adaptive function of autophagy results from a combination of factors, including but not limited to (i) the mobilization of macromolecules including proteins, mRNA, lipids and glycogen to generate energy-rich metabolites and building blocks for anabolic reactions, and (ii) the selective removal of damaged cellular structures including aggregates of misfolded proteins, uncoupled or permeabilized mitochondria, as well as other dysfunctional organelles (Galluzzi et al., 2014; López-Otin and Kroemer, 2021; Mizushima and Klionsky, 2007). As a result, cellular fitness is improved in a cell-autonomous fashion. Moreover, the activation of pro-inflammatory pathways is blunted by autophagy due to the removal of molecules (such as cytosolic DNA or reactive oxygen species) that may activate endogenous pattern recognition receptors, as well as due to the downregulation of the downstream signals emanating from such receptors (Deretic, 2021; Galluzzi et al., 2018; Schwartz, 2021). In view of the broad effects of autophagy, its induction has been proposed as a general strategy to combat diseases.

Recently, an extracellular feedback loop of autophagy was recently described and involves the protein acyl coenzyme A binding protein (ACBP), which is called by diazepam-binding inhibitor (DBI) (Bravo-San Pedro et al., 2019a). Indeed, autophagy is tied to the atypical secretion of this leaderless protein that is predominantly present in the cytosol of nucleated cells (Bravo-San Pedro et al., 2019a; Loomis et al., 2010). Once released into the extracellular space, ACBP/DBI then acts on gamma-aminobutyric acid (GABA) receptors to inhibit autophagy via autocrine, paracrine and neuroendocrine pathways (Bravo-San Pedro et al., 2019a; Joseph et al., 2020). When injected intraperitoneally or intravenously, a monoclonal antibody (mAb) against ACBP/DBI (dubbed as α-DBI) remarkably reduced high-fat diet induced adiposity, diabetes and hepatosteatosis, while enhancing autophagy, lipolysis and β-oxidation and simultaneously reducing appetite (Bravo-San Pedro et al., 2019a; Joseph et al., 2020, 2021). These effects were considered to be on target because they could be mimicked by inducible whole-body knockout of ACBP/DBI (Bravo-San Pedro et al., 2019a). Thus methods and pharmaceutical compositions for modulating autophagy based on the modulation of the activity or expression of DBI were disclosed (WO2019057742).

### SUMMARY OF THE INVENTION

The present invention is defined by the claims. In particular, the present invention relates to methods and pharmaceutical composition of protecting organs from injuries (i.e. treating or reducing tissue damage caused by injury) comprising neutralization of Acyl-CoA Binding Protein.

### DETAILED DESCRIPTION OF THE INVENTION

Acyl-CoA binding protein (ACBP), also known as diazepam-binding inhibitor (DBI), is an extracellular feedback regulator of autophagy. Here, the inventors report that injection of a monoclonal antibody neutralizing ACBP/DBI (α-DBI) (in some embodiments, extracellular DBI) protects (i.e. treats or reduces the incidence of) the murine liver against ischemia/reperfusion damage, acute intoxication by acetaminophen and concanavalin A, as well as against liver fibrosis induced by bile duct ligation or carbon tetrachloride. α-DBI administration downregulated pro-inflammatory and pro-fibrotic genes and upregulated antioxidant defenses and fatty acid oxidation in the liver. Of note, the results support the contention that α-DBI mediates broad organ-protective effects against multiple insults.

### Main Definitions

As used herein, the term “subject”, “individual” or “patient” is used interchangeably and refers to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, the term “organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

As used herein, the term “organ dysfunction” means and includes a reduction or impairment in physical structure or function of the organ.

As used herein, the term “solid organ transplantation” and variations thereof refers to the insertion of a solid organ (also called graft) into a recipient, whether the transplantation is syngeneic (where the donor and recipient are genetically identical), or allogeneic (where the donor and recipient are of different genetic origins but of the same species).

As used herein, the term “insult” or “injury” refers to any damage that directly or indirectly affects the normal functioning. An insult may have a variety of causes including, but not limited to physiological injuries, chemical injuries or physical injuries. The term encompasses acute and chronic injuries. As used herein, the term “acute injury” includes injuries that have recently occurred. For example, an acute injury may have very recently occurred, may have occurred within an hour or less, may have occurred within a day or less, may have occurred within a week or less, or may have occurred within two weeks or less. As used herein, the term “chronic injury” is an injury that has persisted for a period of time. For example, a chronic injury may have occurred more than two weeks ago, may have occurred more than three weeks ago, may have occurred more than two months ago, or may have occurred more than three months ago.

As used herein, the term “ischemia” as used herein refers to a restriction in blood supply with resultant damage or dysfunction of the organ. Rather than hypoxia (a more general term denoting a shortage of oxygen, usually a result of lack of oxygen in the air being breathed), ischemia is an absolute or relative shortage of the blood supply to an organ, i.e. a shortage of oxygen, glucose and other blood-borne components. A relative shortage means the mismatch of blood supply (oxygen/fuel delivery) and blood request for adequate metabolism of tissue. As used herein, the term “warm ischemia” has its general meaning in the art and is used to describe ischemia of cells and tissues under normothermic conditions. As used herein, the term “cold ischemia” has its general meaning in the art and refers to the organ chilling during decreased blood perfusion or in the absence of blood supply.

As used herein, the term “reperfusion” has its general meaning in the art and refers to the restoration of blood flow to a tissue following ischemia. Accordingly, as used herein, the term “ischemia reperfusion” or “I/R” is thus intended to encompass an event wherein an episode of ischemia is followed by an episode of reperfusion.

As used herein, the term “ischemia reperfusion injury” or “I/R injury” refers to the tissue damage caused by an ischemia reperfusion event. The absence of oxygen and nutrients from blood during the ischemic period creates a condition in which the restoration of circulation results in inflammation and oxidative damage through the induction of oxidative stress rather than (or along with) restoration of normal function. As used herein, the term “ischemia reperfusion injury severity” or “I/R injury severity” refers to a measure of the degree of injury.

As used herein, the term “fibrosis” refers to the formation of fibrous tissue as a reparative or reactive process, rather than as a normal constituent of an organ or tissue. Fibrosis is characterized by myofibroblast accumulation and collagen deposition in excess of normal deposition in any particular tissue.

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “polynucleotide sequence encoding an amino acid sequence” includes all nucleotide sequences that are degenerate versions of each other and that encode the same amino acid sequence. The phrase “polynucleotide sequence that encodes a protein or a RNA” may also include introns to the extent that the nucleotide sequence encoding the protein may in some version contain an intron(s).

As used herein, the term “inhibitor of expression” refers to a natural or synthetic compound that has a biological effect to inhibit the expression of a polynucleotide.

As used herein, the term “DBI” has its general meaning in the art and refers to the diazepam binding inhibitor, acyl-CoA binding protein encoding by the DBI gene (Gene ID: 1622). The term is also known as EP; ACBP; ACBD1; and CCK-RP. An exemplary amino acid sequence for DBI is represented by SEQ ID NO: 1.

>sp|P07108|ACBP_HUMAN Acyl-CoA-binding protein
OS = Homo sapiens OX = 9606 GN = DBI PE = 1
SV = 2
SEQ ID NO: 1
MSQAEFEKAAEEVRHLKTKPSDEEMLFIYGHYKQATVGDINTERPGMLD
FTGKAKWDAWNELKGTSKEDAMKAYINKVEELKKKYGI

As used herein the term “antibody” and “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

As used herein the term “human antibody” as used herein, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a KD of about 10⁻⁷ M or less, such as about 10⁻⁸ M or less, such as about 10⁻⁹ M or less, about 10⁻¹⁰ M or less, or about 10⁻¹¹ M or even less.

As used herein, the term “neutralizing anti-DBI monoclonal antibody” refers to an antibody to a monoclonal antibody having specificity for DBI and that inhibits, reduces or completely the activity of DBI (for example, extracellular DBI). Whether an antibody is a neutralizing antibody can be determined by in vitro assays described in the EXAMPLE. Typically, the neutralizing antibody of the present invention inhibits the activity of DBI by at least 50%, 60%, 70%, 80%, 90%, 95%, 99%, or 100%.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, “consisting essentially of”, with reference to a composition, means that the at least one antibody of the invention as described hereinabove is the only one therapeutic agent or agent with a biologic activity within said composition.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of drug are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the patient's size, the severity of the patient's symptoms, and the particular composition or route of administration selected. An exemplary, non-limiting range for a therapeutically effective amount of a drug of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a drug of the present invention is 0.02-100 mg/kg, such as about 0.02-30 mg/kg, such as about 0.05-10 mg/kg or 0.1-3 mg/kg, for example about 0.5-2 mg/kg.

### Methods of Conferring Organ Protection:

The first object of the present invention relates to a method of conferring organ protection (i.e. treating or reducing tissue damage caused by injury) in a subject in need thereof comprising administering to the subject a therapeutically effective amount of an agent that inhibits the activity or expression of DBI (e.g. circulatory or extracellular DBI). As such, disclosed herein is a composition for use in treating or reducing tissue damage caused by an injury described herein in an organ of a subject, the composition comprising an amount of an agent that inhibits the activity or expression of diazepam binding inhibitor (DBI), where the amount is sufficient to treat or reduce the tissue damage caused by the injury to the organ of the subject when administered to the subject.

The method and composition of the present invention is particularly suitable for preventing organ dysfunction.

The method and composition of the present invention is particularly suitable for conferring organ protection against any kind of injury. In some embodiments, the method or composition of the present invention is particularly suitable for conferring organ protection against (treating or reducing tissue damage caused by) chemical injuries. In some embodiments, the method or composition of the present invention is particularly suitable for conferring organ protection against (treating or reducing tissue damage caused by) physical injuries. In some embodiments, the method or composition of the present invention is particularly suitable for conferring organ protection against (treating or reducing tissue damage caused by) ischemic injuries.

In particular, the method or composition of the present invention is particularly suitable for conferring organ protection against (treating or reducing tissue damage caused by) a chemical injury induced by a toxicant selected from the group consisting of alcohol, 2,2′,4,4′,5,5′-hexachlorobiphenyl (PCB-153), 2,3,7,8-tetrachlorodibenzo-p-dioxin (TCDD), 2-bromoethylamine (BEA), 3-methylcholanthrene, 4-aminophenol (PAP), acetaminophen, adriamycin, allyl alcohol, amiodarone, amphotericin B, Aroclor 1254, Aroclor 1260, arsenic, aspirin, astemizole, benzene, cadmium, carbamezipine, carbon tetrachloride (CCl4), ciprofibrate (cipro), clofibrate, cobalt chloride, corvastatin, cyclosporin A, diethylntrosamine, dimethylformamide, dimethylhydrazine (DMH), diquat, ethosuximide, etoposide, famotidine, fluconazole, gamfibrozil, ganciclovir, hexachloro-1,3-butediene (HCBD), HIV protease inhibitors, hydrazine, indomethacin, ketoconazole, lead acetate (PbAc), lipopolysaccharide (LPS), mercury(II) chloride (HgCl 2), methanol, methapyrilene, methotrexate, metronidazole, miconazole, monocrotaline, nitric oxide, ondansetron, pentamidine, phenobarbital, phenylhydrazine (phenylhyrzn), phenytoin, pravastatin, propulsid, puromycin aminonucleoside (PAN), quinolones, simvastatin, sodium fluoride (NaF), statins, thioacetamide, tocainidine, tricyclic antidepressants, troglitazone, tumor necrosis factor α (TNFα), uranyl nitrate, valproic acid, vincristine, Wy-16,463, zidovudine (AZT), α-naphthyl isothiocyanate (ANIT), β-naphthoflavone (BNF), asbestos, radon, cigarette smoke, glues, dioxin, nickel, arsenic, mercury, cement (chromium), polychlorinated biphenyls (PCBs), carbon tetrachloride, methylene chloride, vinyl chloride, mercury, chlorinated hydrocarbon solvents, carbon disulfide, cadmium, ozone, tobacco smoke, nitrates, methylene chloride, ethylene dibromide, and polychlorinated biphenyls.

In some embodiments, the method or composition of the present invention is particularly suitable for conferring organ protection against (treating or reducing tissue damage caused by) a chemotherapeutic agent. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the method of the present invention is particularly suitable for conferring organ protection against (treating or reducing tissue damage caused by) drug-induced injuries induced by an anthracycline selected from the list consisting of: daunorubicin, doxorubicin, epirubicin, farmorubicin, idarubicin, mitoxantrone, pixantrone; and their pharmaceutically acceptable salts.

In some embodiments, the method or composition of the present invention is particularly suitable for the treatment of acetaminophen-induced hepatoxicity, amiodarone-induced pulmonary toxicity, doxorubicin-induced cardiotoxicity, cadmium chloride-induced nephrotoxicity, dimethylnitrosamine-induced spleenotoxicity and O-ethyl-S,S-dipropyl phosphorodithioate (MOCAP)-induced neurotoxicity.

The method or composition of the present invention may be applied to any ischemic insult or event. Tissues that are particularly susceptible to ischemic events include myocardial, vascular and neuronal tissue (particularly cerebral tissue). Other tissues that are susceptible to ischemia include tissue from the gut, liver, kidney and eye. For instance, the need for cardioprotection may arise due to certain physiological disorders such as unstable angina, during trauma or periods of cardiac arrest. In addition, disorders such as stroke, transient ischemic attacks or impending stroke (amarosis fugax) are candidate conditions for treatment using the method of the invention. Where stroke giving rise to a risk of secondary stroke occurs, or another condition giving rise to a risk of stroke within hours or days occurs, the method can be applied to diminish such risk. Those of skill in the art will recognize circumstances associated with increased risk of other ischemic tissue injury. Such disease states include mesenteric artery insufficiency, renal artery stenosis, hepatic vein thrombosis, peripheral vascular insufficiency, multiple trauma, sepsis and multi-organ system failure. Other ischemic events include angiographic evidence of partial coronary artery obstruction, echocardiographic evidence of myocardial damage, or any other evidence of a risk for a future or additional ischemic event (for example a myocardial ischemic event, such as a myocardial infarction (MI), or a neurovascular ischemia such as a cerebrovascular accident CVA). Ischemia/reperfusion may damage tissues other than those of the myocardium. The method or composition provided herein is particularly suitable for reducing ischemia reperfusion injury that can occur in the tissue of the brain, liver, gut, kidney, bowel, or in any other tissue. Additional applications include blunt or penetrating trauma that results in interruption of blood flow to the visceral organs including those arising from penetrating wounds to the abdomen resulting from gunshot wounds, stab wounds or from penetrating wounds or blunt abdominal trauma secondary to deacceleration injury and/or motor vehicle accidents. Other preferred applications include diseases or procedures that result in systemic hypotension that either disrupts or decreases the flow of blood to the visceral organs, including hemorrhagic shock due to blood loss, cardiogenic shock due to myocardial infarction or cardiac failure, neurogenic shock or anaphylaxis.

In some embodiments, the use of the method or composition of the present invention can ameliorate organ protection during surgical procedure requiring stopping of blood supply to an organ followed by reperfusion. Examples of surgical procedures generating a risk of ischemia reperfusion injury include liver resection; revascularization following myocardial infarction, such as by thrombolytic therapy, stenting, or surgical repair; revascularization following stroke, such as by thrombolytic therapy or surgical repair; or revascularization following vascular injury including repair or reattachment of a limb following ischemic injury or surgical repair of an aneurysm. Other examples are surgery of the upper or lower gastrointestinal tract including laparoscopic procedures, open heart surgery with or without heart/lung machine, nose and throat surgery, vascular surgery, neurological (brain) surgery, transplantations (liver, heart, lung, kidney, intestinal), surgeries on the liver and caesarean sections. In some embodiments, the surgical procedure is a Coronary Artery Bypass Surgery, also known as coronary artery bypass graft (CABG) surgery or heart bypass or just bypass surgery which is a surgical procedure performed to relieve angina and reduce the risk of death from coronary artery disease. Arteries or veins from elsewhere in the patient's body are grafted to the coronary arteries to bypass atherosclerotic narrowings and improve the blood supply to the coronary circulation supplying the myocardium (heart muscle). This surgery is usually performed with the heart stopped, necessitating the usage of cardiopulmonary bypass; techniques are available to perform CABG on a beating heart, so-called “off-pump” surgery. In some embodiments, the method of the present invention can be used in any surgical procedure requiring clamping of blood supply to an organ. In particular, the present method of the invention is applied to all surgical procedures, which involve the connection of two blood vessels, e.g., coronary bypass, peripheral bypass, hemodialysis access (creation of a fistula), and free-flap surgery (breast and face reconstruction surgery). More particularly, the method or composition of the present invention may be applied to any surgical procedure that requires anastomosis. The term “anastomosis” as used herein refers to a surgical connection between tubular structures, such as blood vessels.

Typically, the effective amount of the agent that inhibits the activity or expression of DBI (e.g. extracellular DBI) may be administered to the patient before, during or after the reperfusion. In particular, the effective amount of the agent that inhibits the activity or expression of DBI is administered to the patient during the reperfusion of the organ.

In some embodiments, the method or composition of the present invention is particularly suitable for preventing progression to chronic kidney disease (CKD) after an acute kidney injury (AKI). As used herein, the term “chronic kidney disease” (CKD) refers to a progressive loss in renal function over a period of months or years. CKD has its general meaning in the art and is used to classify numerous conditions that affect the kidney, destruction of the renal parenchyma and the loss of functional nephrons or glomeruli. It should be further noted that CKD can result from different causes, but the final pathway remains renal fibrosis. The term “acute kidney injury” or “acute kidney failure” is typically identified by a rapid deterioration in renal function sufficient to result in the accumulation of nitrogenous wastes in the body (see, e.g., Anderson and Schrier (1994), in Harrison's Principles of Internal Medicine, 13th edition, Isselbacher et al, eds., McGraw Hill Text, New York). Rates of increase in BUN of at least 4 to 8 mmol/L/day (10 to 20 mg/dL/day), and rates of increase of serum creatinine of at least 40 to 80 μmolI/L/day (0.5 to 1.0 mg/dL/day), are typical in acute renal failure. Urinary samples also may contain tubular injury residue in patients suffering from acute kidney injury. In subjects which are catabolic (or hypercatabolic), rates of increase in BUN may exceed 100/mg/dL/day. Rates of increase in BUN or serum creatinine may be determined by serial blood tests and, preferably, at least two blood tests are conducted over a period of between 6 and 72 hours or, more preferably, 12 and 24 hours. A distinction is sometimes made between “acute” renal failure (deterioration over a period of days) and “rapidly progressive” renal failure (deterioration over a period of weeks). As used herein, however, the phrase “acute kidney injury” is intended to embrace both syndromes. Acute kidney injury is regularly identified by clinicians, as discussed above. AKI may result from abnormalities of the vasculature such as vasoconstrictive disease (e.g., malignant hypertension, scleroderma, hemolytic uremic syndrome, thrombotic thrombocytopenic purpura) and vasculitis (e.g., polyarteritis nodosa, hypersensitivity anxieties, serum sickness, Wegener's granulomatosis, giant cell arteritis, mixed cryoglobulinemia, Henoch-Schonlein purpura, systemic lupus erythematosus). AKI may also result from abnormalities of the glomeruli such as post-infectious abnormalities (e.g., post-streptococcal, pneumococcal, gonococcal, staphylococcal, enterococci, viral [e.g., hepatitis B and C, mumps, measles, Epstein-Barr], malarial, or related to brucellosis, Legionella, Listeria, shunt nephritis, leprosy, leptospirosis, or visceral abscesses) and non-infectious abnormalities (e.g., rapidly progressive glomerulonephritis, membranoproliferative glomerulonephritis, Goodpasture's syndrome, systemic lupus erythematosus, Wegener's granulomatosis). In some embodiments, AKI may result from acute interstitial nephritis resulting from drug related causes (e.g., penicillins, sulfonamides, carbenicillin, cephalosporin, erythromycin, nafcillin, oxacillin, nonsteroidal antiinflammatory agents, diuretics (furosemide, ethacrynic acid, thiazide, spironolactone, mercurials), phenytoin, phenobarbital, probenicid, allopurinol, cimetidine), infection related causes (e.g., acute pyelonephritis, streptococcal, staphylococcal, leptospirosis, malaria, salmonellosis), papillary necrosis (e.g., associated with diabetes mellitus, sickle cell diseases, analgesic abuse, alcoholism), and other, miscellaneous causes (e.g., sarcoidosis, leukemia, lymphoma). Sime embodiments, AKI may result from intratubular obstruction from crystal deposition (e.g., uric acid, oxalate, methotrexate) or multiple myeloma and light chain disease. In some embodiments, AKI may result from Acute tubular necrosis resulting from nephrotoxins (e.g., antimicrobials such as aminoglycosides, tetracyclines, amphotericin, polymyxin, cephalosporins), heavy metals (e.g., mercury, lead, arsenic, gold salts, barium), and other, miscellaneous chemical agents (e.g., cisplatin, doxorubicin, streptozocin, methoxyflurane, halothane, ethylene glycol, carbon tetrachloride), or from ischemia (e.g., hemorrhage, hypotension, sepsis, burns, renal infarction, renal artery dissection, rhabdomyo lysis, trauma), or other miscellaneous causes (e.g., contrast agents, transfusion reactions, myoglobinemia, heat stroke, snake and spider bites).

The method or composition of the present invention is particularly suitable for preventing, reducing the severity of, or reducing the risk of an injury in an organ. In some embodiments, the organ is isolated. In some embodiments, the organ is a transplant or transplantable organ.

In some embodiments, the method or composition of the present invention can ameliorate organ transplantation by administering to the isolated (transplanted) organ an amount effective of an agent that inhibits the activity or expression of DBI (e.g. extracellular DBI). Accordingly, in some embodiments, the organ is destined to be transplanted in a recipient. The method is thus performed ex vivo in an isolated organ.

In some embodiments, the transplanted organ is a cadaverous organ, and in those instances in which the organ is obtained from a cadaverous donor, the agent that inhibits the activity or expression of DBI can be administered to either the cadaver or the extracted organ. In some embodiments, the transplanted organ is a living organ donation, and in those instances the agent that inhibits the activity or expression of DBI can be administered to the extracted organ.

In some embodiments, the organ is isolated and is perfused with the effective amount of the agent that inhibits the activity or expression of DBI (e.g. extracellular DBI).

In some embodiments, the transplanted organ is the subject of a warm ischemia and/or cold ischemia.

In some embodiments, the effective amount of the agent that inhibits the activity or expression of DBI (e.g. extracellular DBI) is administered during the cold ischemia time. As used herein, the term “cold ischemia time” or “CIT” has its general meaning in the art and refers to the time which extends from the initiation of cold preservation of the recovered organ to restoration of warm circulation after transplantation. There is variability by accepting surgeon/center and by donor and recipient characteristics. Intuitively, shorter CIT is better. For kidney transplantation, the CIT should be inferior to 24 hours; for pancreas transplantation, the CIT should be inferior to 18 hours and for liver transplantation, the CIT should be inferior to 8 hours (Bernat J L, D'Alessandro A M, Port F K, Bleck T P, Heard S O, Medina J, et al. Report of a National Conference on Donation after cardiac death. Am J Transplant. 2006; 6:281-91).

### Methods of Preventing Fibrosis:

The method or composition of the present invention is also particularly suitable for preventing or reducing fibrosis that is associated with or occurs subsequently to an organ injury.

In some embodiments, the fibrosis affects at least one organ selected from the group consisting of in skin, heart, liver, lung, or kidney. Examples of fibrosis include, without limitation, dermal scar formation, keloids, liver fibrosis, lung fibrosis, kidney fibrosis, glomerulosclerosis, pulmonary fibrosis (e.g. idiopathic pulmonary fibrosis), liver fibrosis, renal fibrosis, intestinal fibrosis, interstitial fibrosis, fibrosis of the pancreas and lungs, injection fibrosis, endomyocardial fibrosis, mediastinal fibrosis, myelofibrosis, retroperitoneal fibrosis, progressive massive fibrosis, nephrogenic systemic fibrosis . . . . In some embodiments, the fibrosis is caused by surgical implantation of an artificial organ.

Liver (hepatic) fibrosis, for example, occurs as a part of the wound-healing response to chronic liver injury. Such damage may be the result of viral activity (e.g., chronic hepatitis types B or C) or other infections (e.g., parasites, bacteria), chemicals (e.g., pharmaceuticals, alcohol, pollutants), immune processes (e.g., autoimmune hepatitis), metabolic disorders (e.g., lipid, glycogen, or metal storage disorders), or cancer growth. Liver fibrosis is characterized by the accumulation of extracellular matrix that can be distinguished qualitatively from that in normal liver. Left unchecked, hepatic fibrosis progresses to cirrhosis (defined by the presence of encapsulated nodules), liver failure, and death. A method or composition as described herein cane be provided in an amount sufficient to reduce a level of a transaminase in a subject when administered, as compared to a level of the transaminase in the subject prior to the administering. In some embodiments, the transaminase is aspartate transaminase (AST) or alanine transaminase (ALT). A method or composition as described herein cane be provided in an amount sufficient to reduce a fibrosis score by at least ⅓, relative to a fibrosis score prior to the administering. A method or composition as described herein cane be provided in an amount sufficient to reduce a level of a hydroxyproline in a subject when administered, as compared to a level of hydroxyproline in the subject prior to the administering. A method or composition as described herein cane be provided in an amount sufficient to reduce an NAFLD score in a subject when administered, relative to an NAFLD score prior to the administering.

Fibrotic disorders of the kidney include, without limitation, glomerulonephritis (including membranoproliferative, diffuse proliferative, rapidly progressive, post-infectious, and chronic forms), diabetic glomerulosclerosis, focal glomerulosclerosis, diabetic nephropathy, lupus nephritis, tubulointerstitial fibrosis, membranous nephropathy, amyloidosis (which affects the kidney among other tissues), renal arteriosclerosis, nephrotic syndrome, renal interstitial fibrosis, renal fibrosis in patients receiving cyclosporin, and HIV associated nephropathy. The glomerulus is a major target of many types of renal injury, including immunologic (e.g., immune-complex- or T-cell-mediated), hemodynamic (systemic or renal hypertension), metabolic (e.g., diabetes), “atherosclerotic” (accumulation of lipids in the glomerulus), infiltrative (e.g., amyloid), and toxicant (e.g., snake venom). The renal structural changes in patients with diabetic nephropathy include hypertrophy of the glomerulus, thickening of the glomerular and tubular membranes (due to accumulated matrix), and increased amounts of matrix in the measangium and tubulointerstitium. Glomerular hypertension due to intrarenal hemodynamic changes in diabetes can contribute to the progression of diabetic nephropathy. Autoimmune nephritis can also lead to altered mesangial cell growth responses. Infection by hepatitis-C virus can also result in idiopathic membranoproliferative glomerulonephritis.

Fibrotic disorders of the lung include, without limitation, silicosis, asbestosis, idiopathic pulmonary fibrosis, bronchiolitis obliterans-organizing pneumonia, pulmonary fibrosis associated with high-dose chemotherapy, idiopathic pulmonary fibrosis, and pulmonary hypertension. These diseases are characterized by cell proliferation and increased production of extracellular matrix components, such as collagens, elastin, fibronectin, and tenascin-C. The method of the present invention can also be utilized to treat a subject having asthma and other conditions of the lung associated with airway remodeling.

Pancreatic fibrosis occurs in chronic pancreatitis. This condition is characterized by duct calcification and fibrosis of the pancreatic parenchyma. Like liver cirrhosis, chronic pancreatitis is associated with alcohol abuse.

The method of the present invention is also suitable for the treatment of intestinal fibrosis, particularly fibrosis associated with inflammatory bowel diseases (e.g., Crohn's disease and ulcerative colitis).

Dermal fibrotic conditions which may be treated by the method of the present invention include, but are not limited to, scleroderma, morphea, keloids, hypertrophic scars, familial cutaneous collagenoma, and connective tissue nevi of the collagen type. In addition, the method of the present invention are suitable for inhibiting overproduction of scarring in patients who are known to form keloids or hypertrophic scars, inhibiting or preventing scarring or overproduction of scarring during healing of various types of wounds including surgical incisions, surgical abdominal wounds and traumatic lacerations, preventing or inhibiting scarring and reclosing of arteries following coronary angioplasty, and preventing or inhibiting excess scar or fibrous tissue formation associated with cardiac fibrosis after infarction and in hypersensitive vasculopathy.

Fibrotic conditions of the eye include conditions such as diabetic retinopathy, postsurgical scarring (for example, after glaucoma filtering surgery and after cross-eye surgery), and proliferative vitreoretinopathy.

Fibroproliferative disorders of bone are characterized by aberrant and ectopic bone formation, commonly seen as active proliferation of the major cell types participating in bone formation as well as elaboration by those cells of a complex bone matrix. Exemplary of such bone disorders is the fibrosis that occurs with prostate tumor metastases to the axial skeleton. In prostate tumor-related cancellous bone growth, prostate carcinoma cells can interact reciprocally with osteoblasts to produce enhanced tumor growth and osteoblastic action when they are deposited in bone. Fibroproliferative responses of the bone originating in the skeleton per se include ostepetrosis and hyperstosis. A defect in osteoblast differentiation and function is thought to be a major cause in osteopetrosis, an inherited disorder characterized by bone sclerosis due to reduced bone resorption, marrow cavities fail to develop, resulting in extramedullary hematopoiesis and severe hematologic abnormalities associated with optic atrophy, deafibronectiness, and mental retardation. In osteoarthritis, bone changes are known to occur, and bone collagen metabolism is increased within osteoarthritic femoral heads. The greatest changes occur within the subchondral zone, supporting a greater proportion of osteoid in the diseased tissue.

Fibroproliferative disorders of the vasculature include, for example, transplant vasculopathy, which is a major cause of chronic rejection of heart transplantation. Transplant vasculopathy is characterized by accelerated atherosclerotic plaque formation with diffuse occlusion of the coronary arteries, which is a classic fibroproliferative disease.

Additional fibrotic conditions which may be treated by the method of the present invention include: rheumatoid arthritis, diseases associated with prolonged joint pain and deteriorated joints, progressive systemic sclerosis, polymyositis, dermatomyositis, eosinophilic fascitis, morphea, Raynaud's syndrome, and nasal polyposis.

In some embodiments, the method of the present invention is particularly suitable for the treatment of inflammation-induced fibrosis. As used herein, the expression “inflammation-induced fibrosis” relates to fibrosis developing during inflammatory diseases i.e. diseases related to acute or chronic inflammation (caused by tissue injury, pathogen infections or toxic agents) or as a consequence.

In some embodiments, the method of the present invention is particularly suitable for the treatment of hepatic fibrosis.

### Agents of the Present Invention:

In some embodiments, the agent that inhibits the activity of DBI (e.g. extracellular DBI) is an antibody directed against DBI.

In some embodiments, the antibody is directed against the fragment consisting in the amino acid sequence ranging from the amino acid residue at position 43 to the amino acid residue at position 50 in SEQ ID NO:1 (i.e. the octapeptide or OP).

In some embodiments, the antibody of the present invention is a chimeric antibody, typically a chimeric mouse/human antibody.

In some embodiments, the antibody is a humanized antibody.

In some embodiments, the antibody is a human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody is a neutralizing antibody.

In some embodiments, the neutralizing antibody of the present invention does not mediate antibody-dependent cell-mediated cytotoxicity and thus does not comprise an Fc portion that induces antibody dependent cellular cytotoxicity (ADCC). In some embodiments, the neutralizing antibody does not comprise an Fc domain capable of substantially binding to a FcgRIIIA (CD16) polypeptide. In some embodiments, the neutralizing antibody lacks an Fc domain (e.g. lacks a CH2 and/or CH3 domain) or comprises an Fc domain of IgG2 or IgG4 isotype. In some embodiments, the neutralizing antibody consists of or comprises a Fab, Fab′, Fab′-SH, F (ab′) 2, Fv, a diabody, single-chain antibody fragment, or a multispecific antibody comprising multiple different antibody fragments. In some embodiments, the neutralizing antibody is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by ldusogie et al.

Several anti-DBI antibodies that inhibit the activity of DBI (e.g. extracellular DBI) are suitable for use in the methods and compositions described herein. Such anti-DBI antibodies are commercially available and described in literature. For instance, an antibody that inhibits the activity of DBI (e.g. extracellular DBI) has at least 80%, at least 85%, at least 90%, at least 95%, or has 100% sequence identity to a polypeptide sequence of an antibody selected from the group consisting of: ab231910 (Rabbit polyclonal, abcam), ab232760 (Rabbit polyclonal, abcam), ab16871 (Rabbit polyclonal, abcam), sc-30190 (Rabbit polyclonal, Santa Cruz Biotechnology), FNab02256 (Rabbit polyclonal, Wuhan Fine Biotech Co), PA5-89139 (Rabbit polyclonal, Invitrogen), OTI4A8 (Mouse monoclonal, OnGene), OTI6E12 (Mouse monoclonal, OnGene), or mAb 7A (Mouse monoclonal, Fred Hutch Antibody Technology).

In some embodiments, the agent that inhibits the activity of DBI (e.g. extracellular DBI) is an aptamer directed against DBI. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods (Colas et al., 1996).

In some embodiments, the agent that inhibits the expression of DBI is an inhibitor of expression. In a preferred embodiment of the invention, said inhibitor of gene expression is a siRNA, an endonuclease, an antisense oligonucleotide or a ribozyme.

In some embodiments, the agent that inhibits the activity of DBI (e.g. extracellular DBI) consists in a vaccine composition suitable for eliciting neutralizing autoantibodies against DBI when administered to the subject. For the purpose of the present invention, the term “vaccine composition” is intended to mean a composition which can be administered to humans or to animals in order to induce an immune system response; this immune system response can result in the production of antibodies against DBI. Typically, the vaccine composition comprises at least one antigen derived from DBI. As used herein the term “antigen” refers to a molecule capable of being specifically bound by an antibody or by a T cell receptor (TCR) if processed and presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes). In some embodiments, the antigen consists in a polypeptide comprising an amino acid sequence having at least 80% of identity with the sequence of SEQ ID NO:1 or a fragment thereof (e.g. an epitope). In some embodiments, the antigen consists in a polypeptide comprising i) an amino acid sequence having at least 80% of identity with SEQ ID NO:1, or ii) an amino acid sequence having at least 80% of identity with the amino acid sequence ranging from the amino acid residue at position 17 to the amino acid residue at position 50 in SEQ ID NO:1, or iii) an amino acid sequence having at least 80% of identity with the amino acid sequence ranging from the amino acid residue at position 33 to the amino acid residue at position 50 in SEQ ID NO:1, or iv) an amino acid sequence having at least 80% of identity with the amino acid sequence ranging from the amino acid residue at position 43 to the amino acid residue at position 50 in SEQ ID NO:1. In some embodiments, the polypeptide is conjugated to a carrier protein which is generally sufficiently foreign to elicit a strong immune response to the vaccine. Illustrative carrier proteins are inherently highly immunogenic. Both bovine serum albumin (BSA) and keyhole limpet hemocyanin (KLH) have commonly been used as carriers in the development of conjugate vaccines when experimenting with animals and are contemplated herein as carrier proteins. Proteins which have been used in the preparation of therapeutic conjugate vaccines include, but are not limited to, a number of toxins of pathogenic bacteria and their toxoids. Suitable carrier molecules are numerous and include, but are not limited to: Bacterial toxins or products, for example, cholera toxin B-(CTB), diphtheria toxin, tetanus toxoid, and pertussis toxin and filamentous hemagglutinin, shiga toxin, pseudomonas exotoxin; Lectins, for example, ricin-B subunit, abrin and sweet pea lectin; Sub virals, for example, retrovirus nucleoprotein (retro NP), rabies ribonucleoprotein (rabies RNP), plant viruses (e.g. TMV, cow pea and cauliflower mosaic viruses), vesicular stomatitis virus-nucleocapsid protein (VSV-N), poxvirus vectors and Semliki forest virus vectors; Artificial vehicles, for example, multiantigenic peptides (MAP), microspheres; Yeast virus-like particles (VLPs); Malarial protein antigen; and others such as proteins and peptides as well as any modifications, derivatives or analogs of the above. Other useful carriers include those with the ability to enhance a mucosal response, more particularly, LTB family of bacterial toxins, retrovirus nucleoprotein (retro NP), rabies ribonucleoprotein (rabies RNP), vesicular stomatitis virus-nucleocapsid protein (VSV-N), and recombinant pox virus subunits.

### Pharmaceutical Compositions:

Typically, the agent that inhibits the activity or expression of DBI (e.g. extracellular DBI) is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the agent that inhibits the activity or expression of DBI is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, the agent that inhibits the activity or expression of DBI (e.g. extracellular DBI) of the present invention is administered directly into the subject or isolated organ using injection, pump device and/or any machine (e.g. bypass machine). In some embodiments, an isolated organ suitable for transplantation is perfused with a preservation solution which comprises 5 the effective amount of the agent that inhibits the activity or expression of DBI. As used herein, the terms “preservation solution” or “organ preservation solution” refer to an aqueous solution having a pH between 6.5 and 7.5, including salts, preferably chloride, sulfate, sodium, calcium, magnesium and potassium; sugars, preferably mannitol, raffinose, sucrose, glucose, fructose, lactobionate (which is a water resistant), or gluconate; antioxidants, for instance glutathione; active agents, for instance xanthine oxidase inhibitors such as allopurinol, lactates, amino acids such as histidine, glutamic acid (or glutamate), tryptophan; and optionally colloids such as hydroxyethyl starch, polyethylene glycol or dextran. In some embodiments, a device for preserving an organ is used wherein said device comprises an organ container filled with a preservation solution, characterized in that said device further comprises one or more mean for injecting one or more compound (e.g. the agent that inhibits the activity or expression of DBI) into the organ container.

The invention will be further illustrated by the following figures and examples. However, these examples and figures should not be interpreted in any way as limiting the scope of the present invention.

### FIGURES

FIGS. 1A-1C. Neutralization of ACBP/DBI activates autophagy flux and attenuates organ damage in vivo. FIGS. 1A-FIGS. 1C Liver injury produced by ischemia/reperfusion (IR) for 90 min/4 hours. Mice were pre-treated with i.p. injection of α-DBI or IgG (2.5 μg/g) and HCQ (50 mg/kg) for 4 hours and just before IR. The liver injury (FIG. 1A) was assessed by histological examination. ALT (FIG. 1B) and AST transaminase activity (FIG. 1C) in plasma was analyzed by means of a colorimetric assay (n=5-11 mice per group).

FIGS. 2A-2F. α-DBI alleviates the organ toxicity induced by acetaminophen and concanavalin A in mice. The damage induced by acetaminophen (APAP, i.p. 300 mg/kg for 16 hours) or concanavalin A (ConA, i.v. 12 mg/kg for 4 hours) in mice pre-treated with i.p. injection of α-DBI or IgG (2.5 μg/g) and HCQ (50 mg/kg) for 4 hours and just before hepatic injury.

FIGS. 2A-FIGS. 2C. Hepatoprotective effect of DBI neutralization after APAP intoxication. The hepatic injury (A) was measured by histological examination taking account the area of cell death, degeneration (ballooning), and inflammation around the central veins. ALT and AST transaminases activity (FIG. 2B and FIG. 2C) from plasma mice (n=4-11 mice per group). FIG. 2D-FIG. 2F. Liver protection against ConA-damage by DBI neutralization. Liver injury (FIG. 2D) was scored using grades of infiltration and hepatocyte necrosis. Activity of ALT and AST transaminases (FIGS. 2E and 2F) in plasma (n=3-11 mice per group).

FIGS. 3A-3E. ACBP/DBI neutralization attenuates fibrosis induced by chronic injury. A-C. C57BL/6 mice were subjected to bile duct ligation (BDL) for 2 weeks. Mice were injected with 2.5 μg/g IgG or α-DBI i.p. 4 hours and 1 h before BDL, and twice per week during BDL Fibrosis scores (FIG. 3A), ALT activity (FIG. 3B) and bilirubin levels (FIG. 3C) were measured (n=5-10 mice per group). FIG. 3D-FIG. 3E. C57BL/6 mice were injected i.p. with 2.5 μg/g α-DBI or IgG weekly and 1.6 ml/kg CCl₄ twice per week for 9 weeks. Additional group was treated with 50 mg/kg/day of HCQ for 4 last weeks of CCl₄. Quantification of fibrosis stage (FIG. 3D) and plasma ALT (FIG. 3E) are shown (n=5-14 mice per group).

FIGS. 4A-4D. ACBP/DBI neutralization attenuates hepatosteatosis induced by methionine choline-deficient diet. FIGS. 4A-4D. Male 2-3 months old mice were fed with regular chow diet (RCD) or methionine choline-deficient diet (MCD) for 4 weeks. Hepatic F4/80 macrophage (FIG. 4A), NAFLD activity score (FIG. 4B), ALT and AST activity (FIG. 4C), and p62 levels (FIG. 4D) were measured.

FIGS. 5A-5B. ACBP/DBI neutralization attenuates hepatosteatosis induced by western diet diet. FIGS. 5A-5B. Male 2-3 months old mice were fed with regular chow diet (RCD) or high fat western diet plus sugar for 4 weeks. NAFLD activity score (FIG. 5A) and ALT and AST activity (FIG. 5B) were measured.

FIGS. 6A-6B. ACBP/DBI neutralization attenuates hepatosteatosis induced by western diet diet and CCl₄. FIG. 6A-6B. Male 2-3 months old mice were fed with regular chow diet (RCD) or high fat western diet plus sugar plus CCl₄ for 4 weeks. NAFLD activity score (FIG. 6A) and ALT and AST activity (FIG. 6B) were measured.

FIG. 7. ACBP/DBI neutralization attenuates fibrosis induced by western diet and CCl₄. C57BL/6 mice were fed with regular chow diet (RCD) or high fat western diet plus sugar plus CCl₄ for 4 weeks. The mice were injected i.p. with 2.5 μg/g α-DBI or IgG weekly and 1.6 ml/kg CCl₄ twice per week for 9 weeks. Quantification of fibrosis score is shown (n=5-14 mice per group).

FIGS. 8A-8C. ACBP/DBI neutralization reverses fibrosis induced by CCl₄. Mice received CCl₄ for 9 wk and then were treated with vehicle (oil) for 4 wk of reversion (R). 2.5 μg/g IgG or α-DBI were injected i.p. 1 day before reversion and weekly for R. Quantification of fibrosis stage (FIG. 8A), plasmatic ALT activity (FIG. 8B), and hydroxyproline levels (FIG. 8C) were measured (n=4-12 mice per group).

### EXAMPLE 1: MATERIAL & METHODS

### Chemicals and Reagents

Reagents were obtained from Axon Medchem BV (Groningen, Netherlands), Qiagen (Hilden, Germany), Millipore (MA, USA), Randox (Antrim, UK), Roche Applied Science (Upper Bavaria, Germany) and Sigma Aldrich (MO, USA). Reagents for electrophoresis were obtained from Thermo Fisher Scientific (MA, USA) and BioRad (CA, USA). Antibodies were from Abcam (TX, USA), Abnova (Taipei, Taiwan), Cell Signaling (MA, USA) and Sigma Aldrich.

### Animal Experimentation

Wild-type (Wt) C57BL/6 mice (Envigo, Gannat, France), homozygous Atg4b^{-/-} mice (gift of Dr. Carlos Lopez-Otin, University of Oviedo, Spain), tamoxifen-inducible whole-body knockout of floxed Acbp/Dbi^{-/-} mice (UBC-cre ERT2::Acbp Dbi^{fl/fl}, control: Acbp/Dbi^{fl/fl} without CRE) (Bravo-San Pedro et al., 2019a), homozygous Gabrg2^{mut/mut} mice (bearing a point mutation F771 in the binding site of ACBP/DBI in the gamma-aminobutyric acid A Receptor γ2 subunit) (Wulff et al., 2007), and transgenic mice expressing LC3 conjugated to green fluorescent protein (GFP-LC3-Tg) (Mizushima et al., 2004) were bred and maintained according to the FELASA guidelines and local guidelines from the Animal Experimental Ethics Committee (Permissions #25000, #31411, #34537, #34538, and #34539). Mice were housed in a temperature-controlled environment with 12 h light/dark cycles and were fed with diet and water ad libitum. All animals were sacrificed, and organs were snap-frozen in liquid nitrogen and stored at −80° C., or fixed in 4% buffered paraformaldehyde overnight at 4° C. and embedded in paraffin. Plasma was obtained by cardiac punction.

### Neutralization of DBI by Passive or Active Immunization

The monoclonal antibody against DBI (passive immunization) or isotype IgG (Bioxcell, NH, USA) was used in vivo (2.5 μg/g body weight, B.W., intraperitoneally, i.p., in 200 μL) in a single or several doses. In some experiment, leupeptine (Leu, 30 mg/kg B.W.) was injected i.p. injection 2 h before the end of the experiment.

The production of autoantibodies (active immunization) was induced by conjugation of Keyhole limpet haemocyanin (KLH; from Thermo) and mouse recACBP (KLH-DBI) as Montegut et al. described (Montegut et al., 2022). Briefly, KLH and DBI were mixed at a 1:20 molar ratio and adjusted gradually to 0.25% (v/v) glutaraldehyde. After, the glycine solution was added to finish the reaction, and was ultra-filtrated using a 100 KDa membrane (Millipore). A solution of formaldehyde was added to 0.2% (v/v) final concentration, and the reaction was quenched by addition of a glycine solution followed by an ultrafiltration with 70 mM pH 7.8 phosphate buffer. Male 8-week-old C57BL/6 mice were immunized with i.p. injection of 30, 30, 30, 10 μg of KLH-DBI or KLH alone as an adjuvant emulsion (1:1) with Montanide ISA-51vg (Seppic, Paris, France) on days 0, 7, 14 and 21, respectively.

### Acute Liver Damage in Mice

To induce hepatic ischemia reperfusion injury, male 12-week-old C57BL/6 mice were anesthetized with 2% isofluorane, and a model of segmental (70%) warm hepatic I/R protocol was assessed (Motiño et al., 2019). Briefly, liver ischemia was induced for 90 min, and reperfusion was initiated by removal of the clamp for 4 hours. To induce hepatotoxicity, male 12-week-old C57BL/6 mice were treated with 12 mg/kg Concanavalin A (ConA, Sigma Aldrich) or 300 mg/kg acetaminophen (APAP, Sigma Aldrich) for 4 or 16 hours, respectively. For inhibition of the autophagy flux, the animals were injected i.p. with two doses of 50 mg/kg hydroxychloroquine (HCQ, in PBS; Axon Medchem BV) 4 hours and just before the hepatic damage.

### Hepatic Fibrosis Model In Vivo

To induce fibrosis in the liver, CCl₄ (Sigma Aldrich) was i.p. administered to male 2-months-old C57BL/6 mice at a dose of 1.6 ml/kg twice weekly for 9 weeks (Motiño et al., 2016). Control animals were i.p. injected with the vehicle olive oil (Sigma Aldrich). Additional groups were administrated i.p. with 50 mg/kg HCQ daily for the four last weeks of CCl₄. Another approach to induce hepatic fibrosis involved by bile duct ligation (BDL) for 2 weeks (Tag et al., 2015).

### Biochemical Assays

Serum ALT and AST activity was determined by colorimetric kits (Randox) accordingly with the manufacturer's instructions. To quantify collagen, hepatic hydroxyproline content was assayed by means of a commercial kit (Sigma Aldrich).

### Histopathology

Paraffin-embedded sections (5 μm) were stained with hematoxylin-eosin-safranin (HES) or Sirius Red and were evaluated by experienced pathologist blinded to the features of the animal groups. All slides were scanned with an AxioScan Z1 (Carl Zeiss, Jena, Germany). The NAFLD activity score was assessed using the NAFLD scoring system for mice models validated by Liang et al. (Liang et al., 2014). Briefly, steatosis grade was grouped as follows: grade 0, <5% of steatotic hepatocytes; grade 1, 5-33%; grade 2, 33-66%; and grade 3, >66%. Lobular inflammation was scored as follows: 0, no foci; 1, <2 foci; 2, 2-4 foci; and 3, >4 foci. Ballooning was classified as 0, none; 1, few balloon cells; and 2, many balloon cells. NAFLD activity score was calculated for each liver biopsy based on the sum of scores for steatosis, inflammation, and ballooning. In addition, liver fibrosis staging (Metavir score) was defined as 0, none; 1, perisinusoidal and/or pericentral; 2, incomplete central/central bridging fibrosis; 3, complete central/central bridging fibrosis; and 4, definite cirrhosis (Bedossa and Poynard, 1996). The severity of hepatic IR was graded according to Suzuki's criteria on a scale from 0 to 4. None (0%), minimal (10%), mild (11-30%), moderate (30-60%) and severe (>60%) necrosis, congestion, or centro-lobular ballooning was assigned as grade 0, 1, 2, 3 and 4, respectively (Suzuki et al., 1993). To measure APAP hepatotoxicity, liver samples were classified as none (0; 0%), mild (1; less than 20%), moderate (2; 20 ˜70%,), severe (3; more than 70% of hepatic lobules), taking account of the cell death area, ballooning, and inflammation around the central veins (Naiki-Ito et al., 2010). The hepatic injury induced by ConA was scored using grades as follows: 0, no necrotic infiltrates; 1, small foci of necrotic cells between hepatocytes or necrotic cells surrounding individual hepatocytes; 2, larger foci of 100 necrotic cells or involving 30 hepatocytes; 3, 10% of a hepatic cross-section involved; and 4, 30% of a hepatic cross-section involved (Zhao et al., 2020). Also, to determinate the abundance of hepatic macrophages and Kuppfer cells, liver sections from fixed paraffin blocks were immunohistochemically stained according to standard procedures using anti-mouse F4/80.

### Liver Extracts

For protein or RNA extraction, tissues were homogenized in 2 cycles for 20 s at 5,500 rpm using a Precellys 24 tissue homogenator (Bertin Technologies, Montigny-le-Bretonneux, France) in 20 mM Tris buffer (pH 7.4) containing 150 mM NaCl, 1% Triton X-100, 10 mM EDTA and Complete® protease inhibitor cocktail (Roche Applied Science) or QIAzol (Qiagen), respectively. After, protein extracts were centrifuged at 12,000 g (4° C.) for 15 min and supernatants were collected. Protein concentration in supernatants was evaluated by the bicinchoninic acid technique (BCA protein assay kit, Thermo Fisher Scientific). Homogenate RNA was purified with RNeasy Mini Kit (Qiagen) following the manufacturer's instructions. The purify and concentration of RNA was measured by NanoDrop™ (Thermo Fisher Scientific).

### Data Analysis

Data are expressed as means±SEM. For statistical analysis, firstly, the normal distribution of the results was evaluated by D'Agostino & Pearson normality test and Shapiro-Wilk normality. Statistical significance was analyzed using Student unpaired 2-tailed t-test or unpaired 2-tailed Mann-Whitney test to evaluate the differences between treated and untreated mice within a single genotype and between genotypes. Analysis was performed by using the statistical software GraphPad Prism 5. Whole transcriptome sequencing and GEO datasets statistical analysis was tested by Fisher's exact test. For statistical analysis of metabolomic, the p value was calculated by Mann-Whitney test. All targeted treated data were merged and cleaned with a dedicated R (version 3.4) package (@Github/Kroemerlab/GRMeta). A p<0.05 was considered as statistically significant.

### EXAMPLE 2: ORGAN PROTECTIVE EFFECTS OF ACBP/DBI NEUTRALIZATION AGAINST ACUTE INSULTS

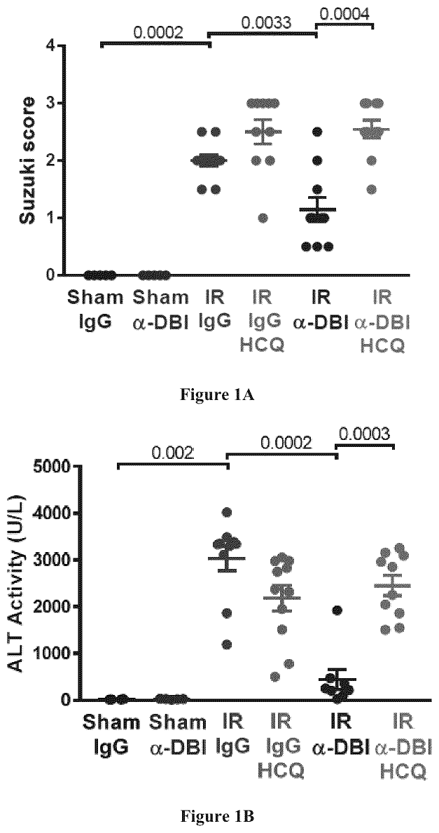

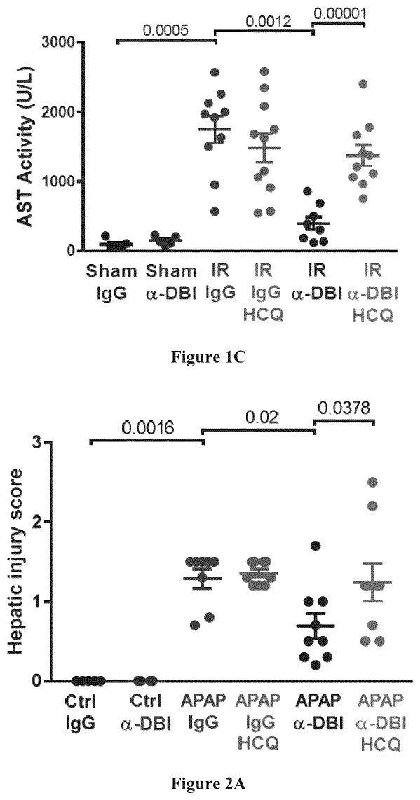

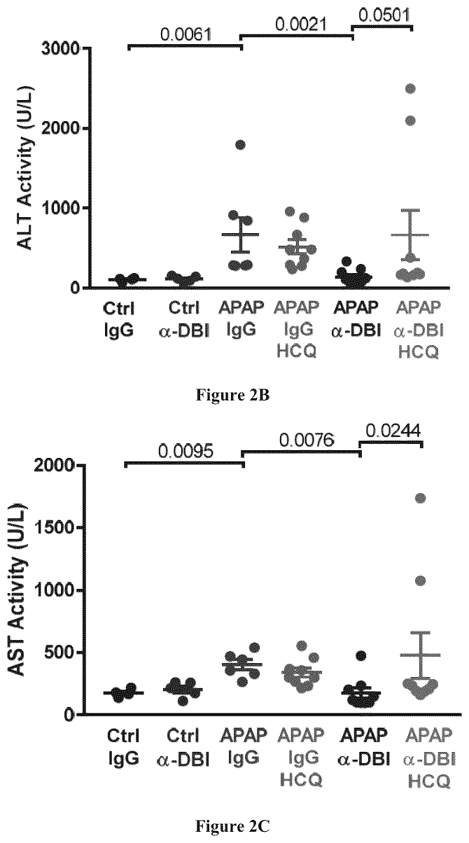

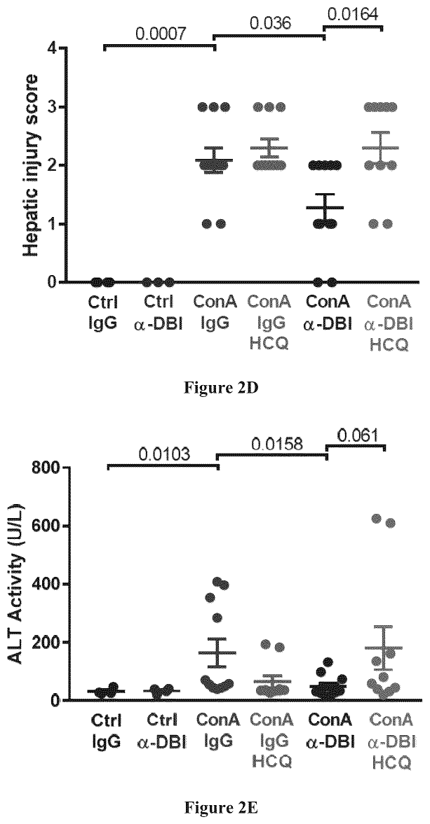

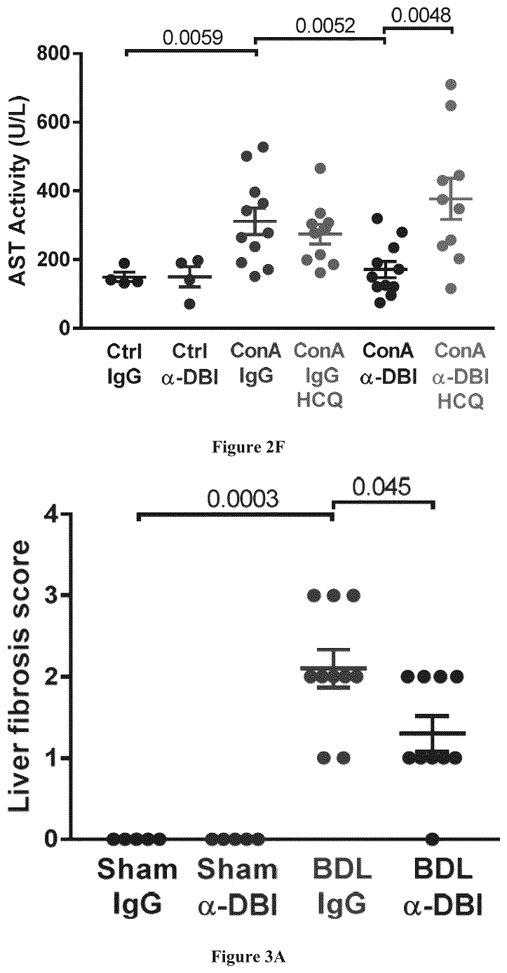

Injection of a monoclonal antibody (mAb) neutralizing ACBP/DBI (α-DBI) (2.5 μg/g intraperitoneally, i.p., 6 and 2 hours before sacrifice) enhances the hepatic lipidation of microtubule-associated proteins 1A/1B light chain 3B (hereafter referred to as LC3B), a marker of autophagy, giving rise to the electrophoretically more mobile LC3-II form (data not shown) (Mizushima et al., 2004). This effect was further enhanced by injection of the lysosomal protease inhibitor leupeptin (30 mg/kg i.p. 2 hours before sacrifice), corroborating the elevated autophagic flux (Haspel et al., 2011) (data not shown). Accordingly, a single injection of α-DBI (2.5 μg/g i.p. 4 hours before sacrifice) induced the formation of autophagic puncta in hepatocytes from mice expressing a transgene encoding a green fluorescent protein (GFP)-LC3 fusion protein (Mizushima et al., 2004) (data not shown). Two injections of α-DBI also reduced the histological signs of ischemia/reperfusion (congestion, ballooning, necrosis summed up in the Suzuki score) (Suzuki et al., 1993) of the liver (FIG. 1A), as well as an increase in the plasma concentrations of the two transaminases alanine aminotransferase (ALT) and aspartate aminotransferase (AST) (FIGS. 1B,1C). When α-DBI injection was combined with hydroxychloroquine (50 mg/kg), a lysomotropic agent that inhibits autophagy in vivo (Cook et al., 2014), the hepatoprotective effects of ACBP/DBI neutralization against ischemia/reperfusion were lost (FIGS. 1A-1C). Similar hydroxychloroquine-inhibitable, hepatoprotective effects of α-DBI were obtained in two models of pharmacological hepatotoxicity caused by acetaminophen (APAP, trade name: paracetamol) and the lectin concanavalin A (ConA) (FIGS. 2A-2F). In both models, α-DBI reduced histological signs of hepatic injury, as well as circulating transaminase levels (FIG. 2D).

### EXAMPLE 3: ACBP/DBI NEUTRALIZATION SUPPRESSES FIBROSIS

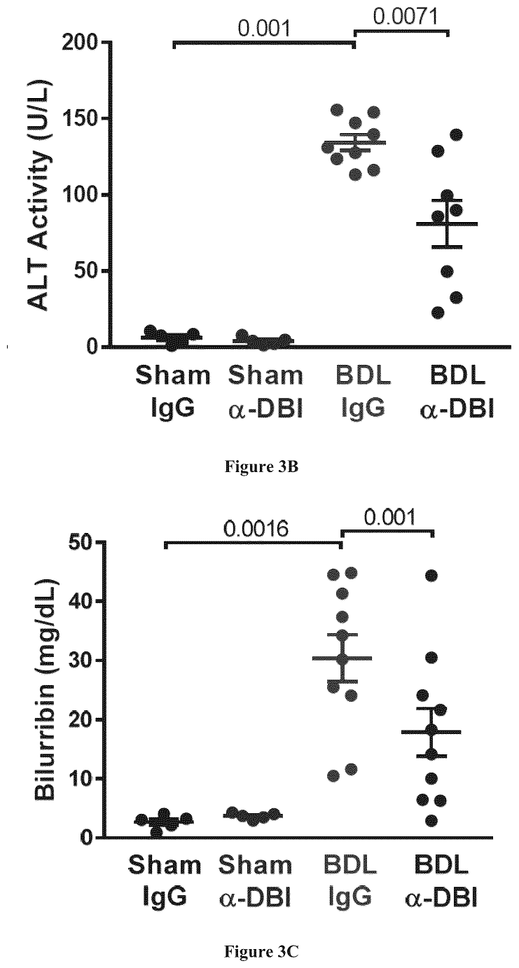

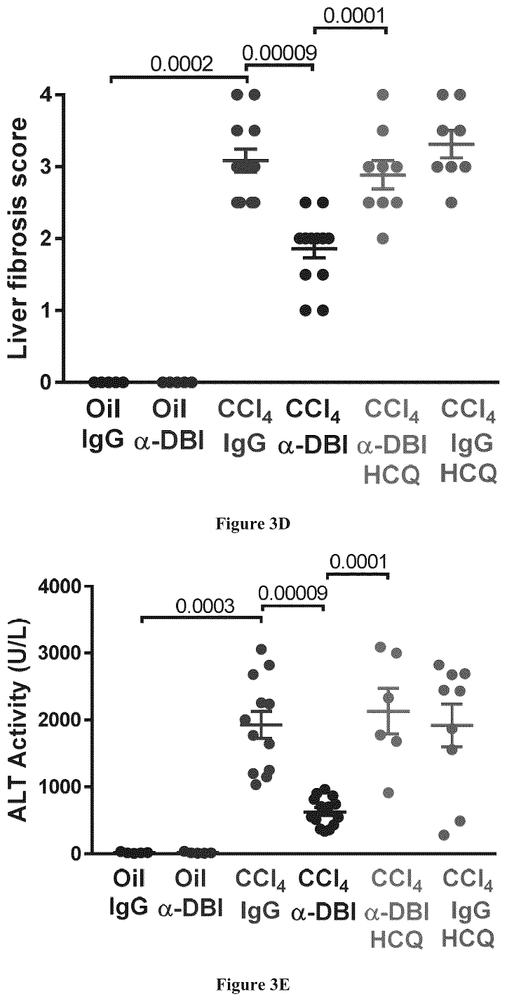

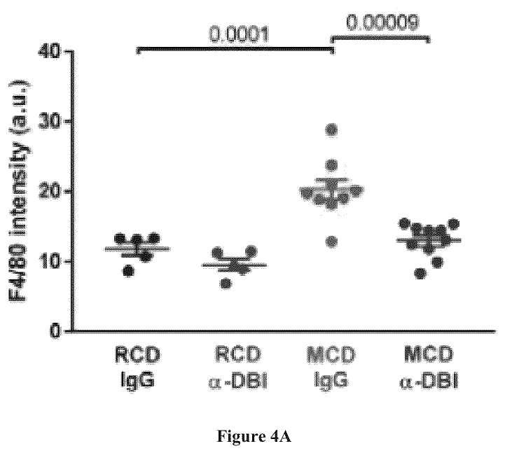

We used a model in which liver fibrosis is induced by bile duct ligation (BLD) (Brea et al., 2018). Two weeks post-BLD, hepatic damage and fibrosis was prominent in isotype control antibody-treated mice, but much attenuated after bi-weekly injection of α-DBI (FIGS. 3A-3C). Similar results were obtained with the well-established model of carbon tetrachloride (CCl₄)-induced liver fibrosis, which was modulated by weekly administration of α-DBI (or isotype IgG control for 9 weeks) and/or daily injections of hydroxychloroquine (or vehicle control during the last 4 weeks of the experiment). In this model, α-DBI attenuated weight loss, signs of fibrosis detectable by Sirius red staining (FIG. 3D) or quantification of the collagen-enriched amino acid hydroxyproline (data not shown), as well as hepatic damage reflected by transaminase activity (FIG. 3E), and by immunoblot detection of the pro-fibrotic markers collagen 1A1 and α-smooth muscle actin (α-SMA) (data not shown). The beneficial effects of α-DBI on liver damage and fibrosis were lost when autophagy was inhibited by hydroxychloroquine (FIGS. 3D,3E). The CCl₄-induced alterations in p62 and LC3-II were reversed by α-DBI, but only in the absence of hydroxychloroquine, not in its presence (data not shown). α-DBI also reversed the CCl₄-induced elevation of circulating transaminases, again only in the absence of hydroxychloroquine (FIG. 3E). Moreover, α-DBI reversed most if not all of the transcriptional effects of chronic CCl₄ intoxication, thus reducing the expression of pro-fibrotic, pro-inflammatory, macrophage associated or transforming growth factor-β (TGF-β)-relevant genes, but enhancing that of anti-oxidant enzymes. These transcriptional effects of α-DBI were abolished when hydroxychloroquine was co-administered (data not shown). In a further set of experiments, we determined whether CCl₄-induced liver fibrosis can be reversed more efficiently when CCl₄ withdrawal is combined with weekly injections of α-DBI for 4 weeks (data not shown). Again, in this curative setting, α-DBI reduced signs of hepatic fibrosis (data not shown). Altogether, these data indicate that ACBP/DBI neutralization has beneficial effects on liver fibrosis that largely depend on autophagy.

### EXAMPLE 4: ACBP/DBI ATTENUATES HEPATOSTEATOSIS INDUCED BY DIET

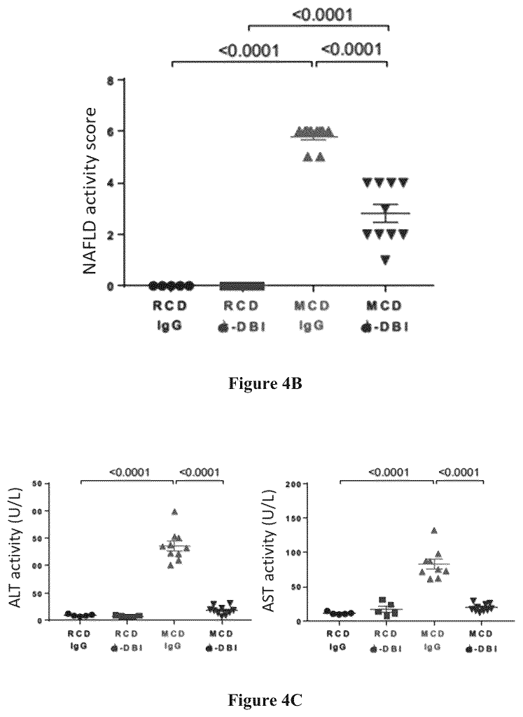

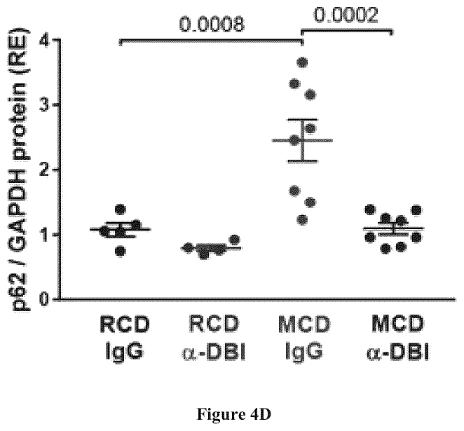

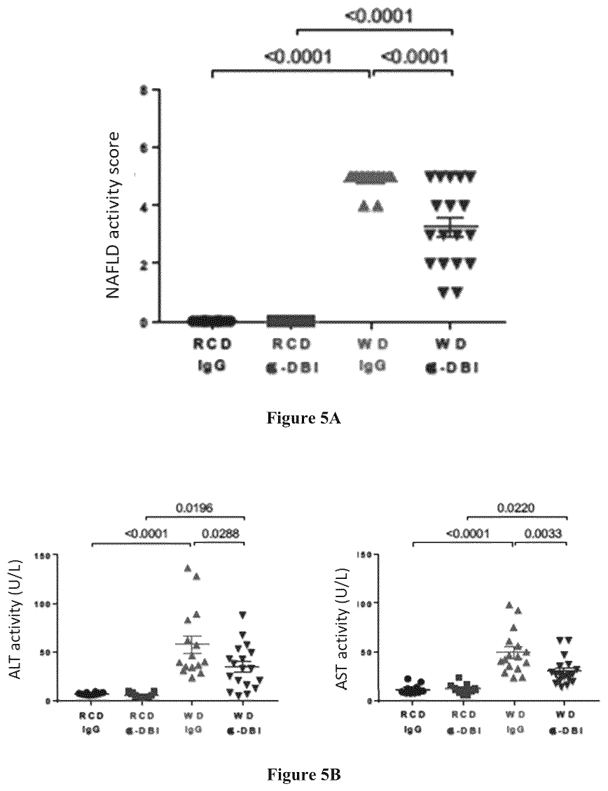

Diet-induced hepatosteatosis was used to model treatment of nonalcoholic fatty liver disease (NAFLD). First, the effects of ACBP/DBI neutralization on a model of NASH that occurs in the context of weight loss, as the result of a methionine choline-deficient diet (MCD) (control: regular chow diet (RCD)). NASH features were evaluated after a 4-wk course of MCD in mice receiving weekly injections of α-DBI (control: isotype immunoglobulin G [IgG]mAb). α-DBI reduced the inflammation associated with NASH, as measured by a reduction in F4/80 macrophage in the liver (FIG. 4A), and largely prevented the histological signs (as determined by NAFLD activity score measured as sum of steatosis, inflammation, and ballooning—FIG. 4B) and enzymological signs (ALT and AST) of NASH induced by MCD (FIG. 4C). Further, α-DBI reversed the depression of autophagic flux associated with NASH, as evidenced by a reduction of the autophagic substrate p62 in mice receiving α-DBI relative to control.

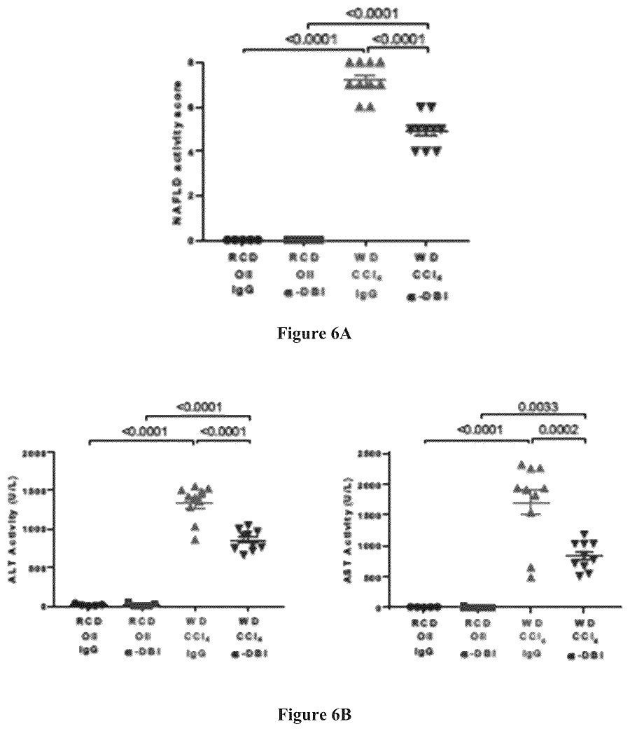

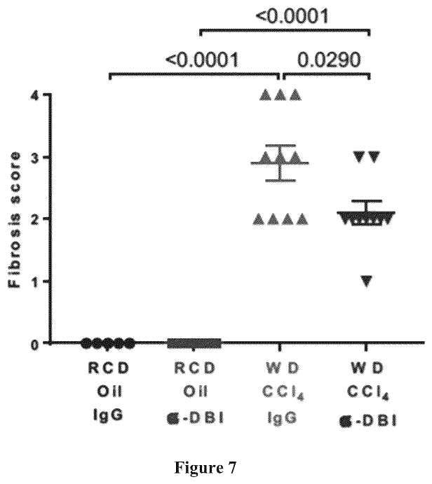

Next, the effects of ACBP/DBI neutralization on a model of NASH that occurs in subjects consuming a high fat Western diet was determined. NASH features were evaluated after a 4-wk course of Western Diet in mice receiving weekly injections of α-DBI (control: isotype immunoglobulin G [IgG]mAb). Further, a separate group of mice receiving Western Diet were administered CCl₄ i.p. in order to induce fibrosis. As with MCD-induced NASH, α-DBI largely prevented the histological signs (as determined by NAFLD activity score—FIG. 5A) and enzymological signs (ALT and AST) of NASH induced by Western Diet (FIG. 5B). Additionally, α-DBI administration reduced NAFLD activity score (FIG. 6A) and enzymological signs (ALT and AST—FIG. 6B) of NASH in the mice receiving Western Diet and administered CCl₄. Furthermore, α-DBI administration reduced the fibrosis associated with CCl₄ administration in the mice receiving Western Diet, as evidenced by a reduction in Fibrosis Score (FIG. 7).

Finally, the ability of α-DBI to provide reversion of liver fibrosis induced by CCl₄ was determined. To induce fibrosis in the liver, CCl₄ (Sigma Aldrich) was i.p. administered to male 2-months-old C57BL/6 mice at a dose of 1.6 ml/kg twice weekly for 9 weeks and then were treated with vehicle (oil) for 4 wk of reversion (R). 2.5 μg/g IgG or α-DBI were injected i.p. 1 day before reversion and weekly for R. Control animals were i.p. injected with the vehicle olive oil (Sigma Aldrich).

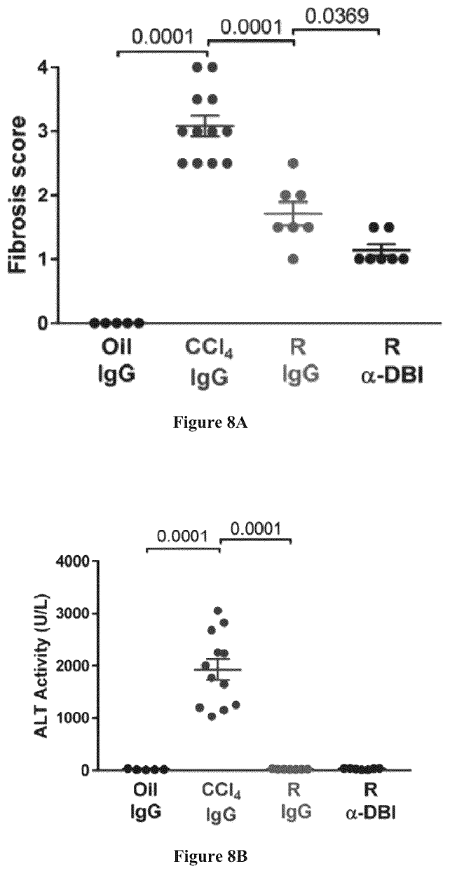

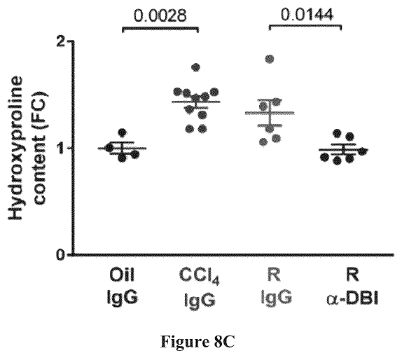

Again, in this curative setting, α-DBI reduced damage signs of hepatic fibrosis, as determined by a reduction in fibrosis score (FIG. 8A), ALT activity (FIG. 8B), and hydroxyproline content (FIG. 8C).

In sum, ACBP/DBI neutralization via α-DBI reduces hepatosteatosis and fibrosis in the liver associated with NASH in various models, and can even reverse fibrosis in the liver after formation, thus further demonstrating the therapeutic potential of ACBP/DBI neutralization via α-DBI.

### REFERENCES

Throughout this application, various references describe the state of the art to which this invention pertains. The disclosures of these references are hereby incorporated by reference into the present disclosure.
Allaire, M., Rautou, P. E., Codogno, P., and Lotersztajn, S. (2019). Autophagy in liver diseases: Time for translation?Journal of Hepatology 70, 985-998.
Aman, Y., Schmauck-Medina, T., Hansen, M., Morimoto, R. I., Simon, A. K., Bjedov, I., Palikaras, K., Simonsen, A., Johansen, T., Tavernarakis, N., et al. (2021). Autophagy in healthy aging and disease. Nature Aging 1, 634-650.
Ashcroft, T., Simpson, J. M., and Timbrell, V. (1988). Simple method of estimating severity of pulmonary fibrosis on a numerical scale. Journal of Clinical Pathology 41, 467.
Bedossa, P., and Poynard, T. (1996). An algorithm for the grading of activity in chronic hepatitis C. Hepatology 24, 289-293.
Bejarano, L., Jordao, M. J. C., and Joyce, J. A. (2021). Therapeutic Targeting of the Tumor Microenvironment. Cancer Discovery 11, 933-959.
Bravo-San Pedro, J. M., Sica, V., Martins, I., Pol, J., Loos, F., Maiuri, M. C., Durand, S., Bossut, N., Aprahamian, F., Anagnostopoulos, G., et al. (2019a). Acyl-CoA-Binding Protein Is a Lipogenic Factor that Triggers Food Intake and Obesity. Cell Metabolism 30, 754-767.e9.
Bravo-San Pedro, J. M., Sica, V., Martins, I., Anagnostopoulos, G., Maiuri, C., and Kroemer, G. (2019b). Cell-autonomous, paracrine and neuroendocrine feedback regulation of autophagy by DBI/ACBP (diazepam binding inhibitor, acyl-CoA binding protein): the obesity factor. Autophagy 15, 2036-2038.
Brea, R., Motiño, O., Frances, D., Garcia-Monzón, C., Vargas, J., Fernández-Velasco, M., Bosch, L., Casado, M., Martin-Sanz, P., and Agra, N. (2018). PGE2 induces apoptosis of hepatic stellate cells and attenuates liver fibrosis in mice by downregulating miR-23a-5p and miR-28a-5p. Biochimica et Biophysica Acta (BBA)—Molecular Basis of Disease 1864, 325-337.
Capurro, M. I., Greenfield, L. K., Prashar, A., Xia, S., Abdullah, M., Wong, H., Zhong, X. Z., Bertaux-Skeirik, N., Chakrabarti, J., Siddiqui, I., et al. (2019). VacA generates a protective intracellular reservoir for Helicobacter pylori that is eliminated by activation of the lysosomal calcium channel TRPML1. Nature Microbiology 4, 1411-1423.
Chao, X., and Ding, W. X. (2019). Role and mechanisms of autophagy in alcohol-induced liver injury. Advances in Pharmacology (San Diego, Calif) 85, 109-131.
Charmpilas, N., Ruckenstuhl, C., Sica, V., Btittner, S., Habemig, L., Dichtinger, S., Madeo, F., Tavernarakis, N., Bravo-San Pedro, J. M., and Kroemer, G. (2020). Acyl-CoA-binding protein (ACBP): a phylogenetically conserved appetite stimulator. Cell Death & Disease 11.
Chiang, W. C., Wei, Y., Kuo, Y. C., Wei, S., Zhou, A., Zou, Z., Yehl, J., Ranaghan, M. J., Skepner, A., Bittker, J. A., et al. (2018). High-Throughput Screens To Identify Autophagy Inducers That Function by Disrupting Beclin 1/Bcl-2 Binding. ACS Chemical Biology 13, 2247-2260.
Choi, M. E. (2020). Autophagy in Kidney Disease. Annual Review of Physiology 82, 297-322.
Cook, K. L., Warri, A., Soto-Pantoja, D. R., Clarke, P. A. G., Cruz, M. I., Zwart, A., and Clarke, R. (2014). Hydroxychloroquine inhibits autophagy to potentiate antiestrogen responsiveness in ER+ breast cancer. Clinical Cancer Research: An Official Journal of the American Association for Cancer Research 20, 3222-3232.
Deretic, V. (2021). Autophagy in inflammation, infection, and immunometabolism. Immunity 54, 437-453.
Dikic, I., and Elazar, Z. (2018). Mechanism and medical implications of mammalian autophagy. Nature Reviews. Molecular Cell Biology 19, 349-364.
Eisenberg, T., Abdellatif, M., Schroeder, S., Primessnig, U., Stekovic, S., Pendl, T., Harger, A., Schipke, J., Zimmermann, A., Schmidt, A., et al. (2016). Cardioprotection and lifespan extension by the natural polyamine spermidine. Nature Medicine 22, 1428-1438.
Fabrizio, P., Hoon, S., Shamalnasab, M., Galbani, A., Wei, M., Giaever, G., Nislow, C., and Longo, V. D. (2010). Genome-wide screen in Saccharomyces cerevisiae identifies vacuolar protein sorting, autophagy, biosynthetic, and tRNA methylation genes involved in life span regulation. PLoS Genetics 6, 1-14.
Fernández, Á.F., Bárcena, C., Martinez-Garcia, G. G., Tamargo-Gómez, I., Suárez, M. F., Pietrocola, F., Castoldi, F., Esteban, L., Sierra-Filardi, E., Boya, P., et al. (2017). Autophagy couteracts weight gain, lipotoxicity and pancreatic β-cell death upon hypercaloric pro-diabetic regimens. Cell Death & Disease 8.
Fernández, Á. F., Sebti, S., Wei, Y., Zou, Z., Shi, M., McMillan, K. L., He, C., Ting, T., Liu, Y., Chiang, W. C., et al. (2018). Disruption of the beclin 1-BCL2 autophagy regulatory complex promotes longevity in mice. Nature 558, 136-140.
Galluzzi, L., Pietrocola, F., Levine, B., and Kroemer, G. (2014). Metabolic control of autophagy. 5 Cell 159, 1263-1276.
Galluzzi, L., Bravo-San Pedro, J. M., Levine, B., Green, D. R., and Kroemer, G. (2017). Pharmacological modulation of autophagy: therapeutic potential and persisting obstacles. Nature Reviews. Drug Discovery 16, 487-511.
Galluzzi, L., Yamazaki, T., and Kroemer, G. (2018). Linking cellular stress responses to systemic homeostasis. Nature Reviews. Molecular Cell Biology 19, 731-745.
Grajeda-lglesias, C., Durand, S., Daillere, R., Iribarren, K., Lemaitre, F., Derosa, L., Aprahamian, F., Bossut, N., Nirmalathasan, N., Madeo, F., et al. (2021). Oral administration of Akkermansia muciniphila elevates systemic antiaging and anticancer metabolites. Aging 13, 6375-6405.
Hansen, M., Rubinsztein, D. C., and Walker, D. W. (2018). Autophagy as a promoter of longevity: insights from model organisms. Nature Reviews. Molecular Cell Biology 19, 579-593.
Haspel, J., Shaik, R. S., Ifedigbo, E., Nakahira, K., Dolinay, T., Englert, J. A., and Choi, A. M. K. (2011). Characterization of macroautophagic flux in vivo using a leupeptin-based assay. Autophagy 7, 629-642.
Hazari, Y., Bravo-San Pedro, J. M., Hetz, C., Galluzzi, L., and Kroemer, G. (2020). Autophagy in hepatic adaptation to stress. Journal of Hepatology 72, 183-196.
Joseph, A., Moriceau, S., Sica, V., Anagnostopoulos, G., Pol, J., Martins, I., Lafarge, A., Maiuri, M. C., Leboyer, M., Loftus, J., et al. (2020). Metabolic and psychiatric effects of acyl coenzyme A binding protein (ACBP)/diazepam binding inhibitor (DBI). Cell Death & Disease 11.
Joseph, A., Chen, H., Anagnostopoulos, G., Montégut, L., Lafarge, A., Motiño, O., Castedo, M., Maiuri, M. C., Clement, K., Terrisse, S., et al. (2021). Effects of acyl-coenzyme A binding protein (ACBP)/diazepam-binding inhibitor (DBI) on body mass index. Cell Death & Disease 12.
Kim, D., Paggi, J. M., Park, C., Bennett, C., and Salzberg, S. L. (2019). Graph-based genome alignment and genotyping with HISAT2 and HISAT-genotype. Nature Biotechnology 37, 907-915.
Kim, S. N., Lee, J., Yang, H. S., Cho, J. W., Kwon, S., Kim, Y. B., Her, J. D., Cho, K. H., Song, C. W., and Lee, K. (2010). Dose-response Effects of Bleomycin on Inflammation and Pulmonary Fibrosis in Mice. Toxicological Research 26, 217.
Kitada, M., and Koya, D. (2021). Autophagy in metabolic disease and ageing. Nature Reviews. Endocrinology 17, 647-661.
Klionsky, D. J., Petroni, G., Amaravadi, R. K., Baehrecke, E. H., Ballabio, A., Boya, P., Bravo-San Pedro, J. M., Cadwell, K., Cecconi, F., Choi, A. M. K., et al. (2021). Autophagy in major human diseases. The EMBO Journal 40.
Kroemer, G., and Levine, B. (2008). Autophagic cell death: the story of a misnomer. Nature 5 Reviews. Molecular Cell Biology 9, 1004-1010.
Levine, B., and Kroemer, G. (2019). Biological Functions of Autophagy Genes: A Disease Perspective. Cell 176, 11-42.
Liang, W., Menke, A. L., Driessen, A., Koek, G. H., Lindeman, J. H., Stoop, R., Havekes, L. M., Kleemann., R., and van den Hoek, A. M. (2014). Establishment of a general NAFLD scoring system for rodent models and comparison to human liver pathology. PloS One 9.
Lin, Z., Wu, F., Lin, S., Pan, X., Jin, L., Lu, T., Shi, L., Wang, Y., Xu, A., and Li, X. (2014). Adiponectin protects against acetaminophen-induced mitochondrial dysfunction and acute liver injury by promoting autophagy in mice. Journal of Hepatology 61, 825-831.
Loomis, W. F., Behrens, M. M., Williams, M. E., and Anjard, C. (2010). Pregnenolone sulfate and cortisol induce secretion of acyl-CoA-binding protein and its conversion into endozepines from astrocytes. The Journal of Biological Chemistry 285, 21359-21365.
López-Otin, C., and Kroemer, G. (2021). Hallmarks of Health. Cell 184, 33-63.
Love, M. I., Huber, W., and Anders, S. (2014). Moderated estimation of fold change and dispersion for RNA-seq data with DESeq2. Genome Biology 15.
Lu, Y. X., Regan, J. C., EBer, J., Drews, L. F., Weinseis, T., Stinn, J., Hahn, O., Miller, R. A., Grönke, S., and Partridge, L. (2021). A TORCl-histone axis regulates chromatin organisation and non-canonical induction of autophagy to ameliorate ageing. ELife 10.
Madeo, F., Carmona-Gutierrez, D., Hofer, S. J., and Kroemer, G. (2019). Caloric Restriction Mimetics against Age-Associated Disease: Targets, Mechanisms, and Therapeutic Potential. Cell Metabolism 29, 592-610.
Meléndez, A., Tallóczy, Z., Seaman, M., Eskelinen, E. L., Hall, D. H., and Levine, B. (2003). Autophagy genes are essential for dauer development and life-span extension in C. elegans. Science (New York, N. Y.) 301, 1387-1391.
Mizushima, N., and Klionsky, D. J. (2007). Protein turnover via autophagy: implications for metabolism. Annual Review of Nutrition 27, 19-40.
Mizushima, N., Yamamoto, A., Matsui, M., Yoshimori, T., and Ohsumi, Y. (2004). In vivo analysis of autophagy in response to nutrient starvation using transgenic mice expressing a fluorescent autophagosome marker. Molecular Biology of the Cell 15, 1101-1111.
Montagner, A., Polizzi, A., Fouche, E., Ducheix, S., Lippi, Y., Lasserre, F., Barquissau, V., Regnier, M., Lukowicz, C., Benhamed, F., et al. (2016). Liver PPARa is crucial for whole-body fatty acid homeostasis and is protective against NAFLD. Gut 65, 1202-1214.
Montégut, L., Chen, H., Bravo-San Pedro, J. M., Motiño, O., Martins, I., and Kroemer, G. (2022). Immunization of mice with the self-peptide ACBP coupled to keyhole limpet hemocyanin. STAR Protocols 3, 101095.
Morishita, H., and Mizushima, N. (2019). Diverse Cellular Roles of Autophagy. Annual Review of Cell and Developmental Biology 35, 453-475.
Motiño, O., Agra, N., Brea Contreras, R., Dominguez-Moreno, M., Garcia-Monzón, C., Vargas-Castrillón, J., Carnovale, C. E., Bosch, L., Casado, M., Mayoral, R., et al. (2016). Cyclooxygenase-2 expression in hepatocytes attenuates non-alcoholic steatohepatitis and liver fibrosis in mice. Biochimica et Biophysica Acta—Molecular Basis of Disease 1862.
Motiño, O., Francés, D. E., Casanova, N., Fuertes-Agudo, M., Cucarella, C., Flores, J. M., Vallejo-Cremades, M. T., Olmedilla, L., Pérez Peña, J., Bañares, R., et al. (2019). Protective Role of Hepatocyte Cyclooxygenase-2 Expression Against Liver Ischemia-Reperfusion Injury in Mice. Hepatology 70.
Naiki-Ito, A., Asamoto, M., Naiki, T., Ogawa, K., Takahashi, S., Sato, S., and Shirai, T. (2010). Gap junction dysfunction reduces acetaminophen hepatotoxicity with impact on apoptotic signaling and connexin 43 protein induction in rat. Toxicologic Pathology 38, 280-286.
Nakade, Y., Sakamoto, K., Yamauchi, T., Inoue, T., Kobayashi, Y., Yamamoto, T., Ishii, N., Ohashi, T., Sumida, Y., Ito, K., et al. (2017). Conophylline inhibits non-alcoholic steatohepatitis in mice. PloS One 12.
Neess, D., Bek, S., Engelsby, H., Gallego, S. F., and Forgeman, N. J. (2015). Long-chain acyl-CoA esters in metabolism and signaling: Role of acyl-CoA binding proteins. Progress in Lipid Research 59, 1-25.
Neess, D., Kruse, V., Marcher, A. B., Wxde, M. R., Vistisen, J., Moller, P. M., Petersen, R., Brewer, J. R., Ma, T., Colleluori, G., et al. (2021). Epidermal Acyl-CoA-binding protein is indispensable for systemic energy homeostasis. Molecular Metabolism 44.
Powell, E. E., Wong, V. W. S., and Rinella, M. (2021). Non-alcoholic fatty liver disease. Lancet (London, England) 397, 2212-2224.
Pyo, J. O., Yoo, S. M., Ahn, H. H., Nah, J., Hong, S. H., Kam, T. I., Jung, S., and Jung, Y. K. (2013). Overexpression of Atg5 in mice activates autophagy and extends lifespan. Nature Communications 4.
Racanelli, A. C., Choi, A. M. K., and Choi, M. E. (2020). Autophagy in chronic lung disease. Progress in Molecular Biology and Translational Science 172, 135-156.
Schlaepfer, I. R., and Joshi, M. (2020). CPT1A-mediated Fat Oxidation, Mechanisms, and Therapeutic Potential. Endocrinology 161.
Schwartz, L. M. (2021). Autophagic Cell Death During Development—Ancient and Mysterious. Frontiers in Cell and Developmental Biology 9.
Sciarretta, S., Maejima, Y., Zablocki, D., and Sadoshima, J. (2018a). The Role of Autophagy in the Heart. Annual Review of Physiology 80, 1-26.
Sciarretta, S., Yee, D., Nagarajan, N., Bianchi, F., Saito, T., Valenti, V., Tong, M., del Re, D. P., Vecchione, C., Schirone, L., et al. (2018b). Trehalose-Induced Activation of Autophagy Improves Cardiac Remodeling After Myocardial Infarction. Journal of the American College of Cardiology 71, 1999-2010.
Serviddio, G., Giudetti, A. M., Bellanti, F., Priore, P., Rollo, T., Tamborra, R., Siculella, L., Vendemiale, G., Altomare, E., and Gnoni, G. v. (2011). Oxidation of Hepatic Carnitine Palmitoyl Transferase-I (CPT-I) Impairs Fatty Acid Beta-Oxidation in Rats Fed a Methionine-Choline Deficient Diet. PLoS ONE 6, 24084.
Shamalnasab, M., Dhaoui, M., Thondamal, M., Harvald, E. B., Forgeman, N. J., Aguilaniu, H., and Fabrizio, P. (2017). HIF-1-dependent regulation of lifespan in Caenorhabditis elegans by the acyl-CoA-binding protein MAA-1. Aging 9, 1745-1769.
Sharma, P., and Allison, J. P. (2015). Immune checkpoint targeting in cancer therapy: toward combination strategies with curative potential. Cell 161, 205-214.
Shen, Y., Malik, S. A., Amir, M., Kumar, P., Cingolani, F., Wen, J., Liu, Y., Zhao, E., Farris, A. B., Raeman, R., et al. (2020). Decreased Hepatocyte Autophagy Leads to Synergistic IL-1β and TNF Mouse Liver Injury and Inflammation. Hepatology (Baltimore, Md.) 72, 595-608.
Shirakabe, A., Fritzky, L., Saito, T., Zhai, P., Miyamoto, S., Gustafsson, A. B., Kitsis, R. N., and Sadoshima, J. (2016). Evaluating mitochondrial autophagy in the mouse heart. Journal of Molecular and Cellular Cardiology 92, 134-139.
Suzuki, S., Toledo-Pereyra, L. H., Rodriguez, F. J., and Cejalvo, D. (1993). Neutrophil infiltration as an important factor in liver ischemia and reperfusion injury. Modulating effects of FK506 and cyclosporine. Transplantation 55, 1265-1271.
Tag, C. G., Sauer-Lehnen, S., Weiskirchen, S., Borkham-Kamphorst, E., Tolba, R. H., Tacke, F., and Weiskirchen, R. (2015). Bile Duct Ligation in Mice: Induction of Inflammatory Liver Injury and Fibrosis by Obstructive Cholestasis. JoVE (Journal of Visualized Experiments) 96, e52438.
Tavernarakis, N., Pasparaki, A., Tasdemir, E., Maiuri, M. C., and Kroemer, G. (2008). The effects of p53 on whole organism longevity are mediated by autophagy. Autophagy 4, 870-873.
Thomas, A. M., Asnicar, F., Kroemer, G., and Segata, N. (2021). Genes Encoding Microbial Acyl Coenzyme A Binding Protein/Diazepam-Binding Inhibitor Orthologs Are Rare in the Human Gut Microbiome and Show No Links to Obesity. Applied and Environmental Microbiology 87, 1-11.
Tong, M., Saito, T., Zhai, P., Oka, S. I., Mizushima, W., Nakamura, M., Ikeda, S., Shirakabe, A., and Sadoshima, J. (2019). Mitophagy Is Essential for Maintaining Cardiac Function During High Fat Diet-Induced Diabetic Cardiomyopathy. Circulation Research 124, 1360-1371.
Venkatesh, S., Li, M., Saito, T., Tong, M., Rashed, E., Mareedu, S., Zhai, P., Bircena, C., López-Otin, C., Yehia, G., et al. (2019). Mitochondrial LonP1 protects cardiomyocytes from ischemia/reperfusion injury in vivo. Journal of Molecular and Cellular Cardiology 128, 38-50.
Wang, J., Vasaikar, S., Shi, Z., Greer, M., and Zhang, B. (2017). WebGestalt 2017: a more comprehensive, powerful, flexible and interactive gene set enrichment analysis toolkit. Nucleic Acids Research 45, W130-W137.
Wulff, P., Goetz, T., Leppa, E., Linden, A. M., Renzi, M., Swinny, J. D., Vekovischeva, O. Y., Sieghart, W., Somogyi, P., Korpi, E. R., et al. (2007). From synapse to behavior: rapid modulation of defined neuronal types with engineered GABAA receptors. Nature Neuroscience 10, 923-929.
Xiao, S., and Chye, M. L. (2010). The Arabidopsis thaliana ACBP3 regulates leaf senescence by modulating phospholipid metabolism and ATG8 stability. Autophagy 6.
Zhao, J., Park, S., Kim, J. W., Qi, J., Zhou, Z., Lim, C. W., and Kim, B. (2020). Nicotine attenuates concanavalin A-induced liver injury in mice by regulating the α7-nicotinic acetylcholine receptor in Kupffer cells. International Immunopharmacology 78.

1. A method of treating tissue damage caused by chemical injury, physical injury, or ischemic injury in an organ of a subject in need thereof, the method comprising administering to the subject a therapeutically effective amount of an agent that inhibits the activity or expression of diazepam binding inhibitor (DBI), wherein the administering is sufficient to treat the tissue damage in the organ of the subject caused by chemical injury, physical injury, or ischemic injury.

2. The method of claim 1, wherein the agent inhibits the activity of extracellular DBI.

3. The method of claim 1, wherein the tissue damage is caused by chemical injury induced by a toxicant selected from the group consisting of alcohol, 2,2′,4,4′,5,5′-hexachlorobiphenyl (PCB-153), 2,3,7,8-tetrachlorodibenzo-p-dioxin (TCDD), 2-bromoethylamine (BEA), 3-methylcholanthrene, 4-aminophenol (PAP), acetaminophen, adriamycin, allyl alcohol, amiodarone, amphotericin B, Aroclor 1254, Aroclor 1260, arsenic, aspirin, astemizole, benzene, cadmium, carbamezipine, carbon tetrachloride (CCl₄), ciprofibrate (cipro), clofibrate, cobalt chloride, corvastatin, cyclosporin A, diethylntrosamine, dimethylformamide, dimethylhydrazine (DMH), diquat, ethosuximide, etoposide, famotidine, fluconazole, gamfibrozil, ganciclovir, hexachloro-1,3-butediene (HCBD), HIV protease inhibitors, hydrazine, indomethacin, ketoconazole, lead acetate (PbAc), lipopolysaccharide (LPS), mercury(II) chloride (HgCl 2), methanol, methapyrilene, methotrexate, metronidazole, miconazole, monocrotaline, nitric oxide, ondansetron, pentamidine, phenobarbital, phenylhydrazine (phenylhyrzn), phenytoin, pravastatin, propulsid, puromycin aminonucleoside (PAN), quinolones, simvastatin, sodium fluoride (NaF), statins, thioacetamide, tocainidine, tricyclic antidepressants, troglitazone, tumor necrosis factor α (TNFα), uranyl nitrate, valproic acid, vincristine, Wy-16,463, zidovudine (AZT), α-naphthyl isothiocyanate (ANIT), β-naphthoflavone (BNF), asbestos, radon, cigarette smoke, glues, dioxin, nickel, arsenic, mercury, cement (chromium), polychlorinated biphenyls (PCBs), carbon tetrachloride, methylene chloride, vinyl chloride, mercury, chlorinated hydrocarbon solvents, carbon disulfide, cadmium, ozone, tobacco smoke, nitrates, methylene chloride, ethylene dibromide, and polychlorinated biphenyls.

4. The method of claim 1, wherein the tissue damage caused by a chemotherapeutic agent.

5. The method of claim 4, wherein the chemotherapeutic agent is an anthracycline selected from the group consisting of: daunorubicin, doxorubicin, epirubicin, farmorubicin, idarubicin, mitoxantrone, pixantrone; and their pharmaceutically acceptable salts.

6. The method of claim 1, wherein the agent that inhibits the activity or expression of DBI is present in an amount that is sufficient to treat acetaminophen-induced hepatoxicity, amiodarone-induced pulmonary toxicity, doxorubicin-induced cardiotoxicity, cadmium chloride-induced nephrotoxicity, dimethylnitrosamine-induced spleenotoxicity or O-ethyl-S,S-dipropyl phosphorodithioate (MOCAP)-induced neurotoxicity.

7. The method of claim 1, wherein the tissue damage is caused by ischemic injury.

8. The method of claim 7, wherein the ischemic injury is a myocardial infarction or an ischemia reperfusion injury.

9. The method of claim 1, wherein the agent that inhibits the activity or expression of DBI is present in an amount that is sufficient to reduce or delay the progression of an acute kidney injury (AKI) to chronic kidney disease (CKD).

10. A method of reducing the severity of an injury in an isolated organ, the method comprising contacting the isolated organ with an agent that inhibits the activity or expression of diazepam binding inhibitor (DBI) in an amount sufficient to reduce the severity of the injury in the isolated organ.

11. The method of claim 10, wherein the isolated organ is a transplantable organ.

12. A method of reducing fibrosis associated with an organ injury in a subject in need thereof, the method comprising administering to the subject a therapeutically effect amount of an agent that inhibits the activity or expression of diazepam binding inhibitor (DBI), wherein the administering is sufficient to reduce an amount of the fibrosis associated with the organ injury in an organ of the subject, as compared to an amount of fibrosis in the organ in the absence of the administrating.

13. The method of claim 12, wherein the agent inhibits the activity of extracellular DBI.

14. The method of claim 12, wherein the fibrosis occurs in an organ selected from the group consisting of: skin, heart, liver, lung, and kidney.

15. The method of claim 12, wherein the agent that inhibits the activity or expression of DBI is present in an amount that is sufficient to reduce a level of a transaminase in the subject, as compared to a level of the transaminase in the subject prior to the administering.

16. The method of claim 15, wherein the transaminase is aspartate transaminase (AST) or alanine transaminase (ALT).

17. The method of claim 12, wherein the agent that inhibits the activity or expression of DBI is present in an amount that is sufficient to reduce a fibrosis score by at least ⅓, relative to a fibrosis score prior to the administering.

18. The method of claim 12, wherein the agent that inhibits the activity or expression of DBI is present in an amount that is sufficient to reduce a level of a hydroxyproline in the subject, as compared to a level of hydroxyproline in the subject prior to the administering.

19. The method of claim 12, wherein the fibrosis is associated with nonalcoholic fatty liver disease (NAFLD).

20. The method of claim 12, wherein the agent that inhibits the activity or expression of DBI is present in an amount that is sufficient to reduce an NAFLD score in the subject, as compared to an NAFLD score prior to the administering.

21. The method of claim 1, wherein the agent that inhibits the activity of DBI is an antibody or an aptamer directed against DBI.

22. The method of claim 21, wherein the antibody is directed against a polypeptide fragment of DBI that comprises the amino acid sequence from position 43 to position 50 of SEQ ID NO: 1.

23. The method of claim 21, wherein the antibody is a monoclonal chimeric antibody, a monoclonal humanised antibody, or a monoclonal human antibody.

24. The method of claim 1, wherein the agent that inhibits the expression of DBI is an inhibitor of expression selected from the group consisting of: an siRNA, an endonuclease, an antisense oligonucleotide, and a ribozyme.

25. The method of claim 1, wherein the agent that inhibits the activity of DBI is present as a vaccine composition suitable for eliciting neutralizing autoantibodies against DBI when administered to the subject.

26. The method of claim 25, wherein the vaccine composition comprises an antigen that comprises i) an amino acid sequence having at least 80% identity to SEQ ID NO:1, ii) an amino acid sequence having at least 80% identity to the amino acid sequence from position 17 to position 50 of SEQ ID NO: 1, iii) an amino acid sequence having at least 80% identity to the amino acid sequence from position 33 to position 50 of SEQ ID NO: 1, or iv) an amino acid sequence having at least 80% identity to the amino acid sequence from position 43 to position 50 of SEQ ID NO: 1.

27. A method of treating tissue damage caused by chemical injury, physical injury, or ischemic injury in a liver or a heart of a subject in need thereof, comprising administering to the subject a therapeutically effective amount of an agent that inhibits the activity or expression of diazepam binding inhibitor (DBI), wherein the administering is sufficient to treat tissue damage in the liver or the heart of the subject caused by the chemical injury, physical injury, or ischemic injury.
